(19) United States
(12) Patent Application Publication
(10) Pub. No.: US 2026/0048155 A1
(43) Pub. Date: Feb. 19, 2026

(54) BIOCOMPATIBLE PARAHYDROGEN HYPERPOLARIZED SOLUTIONS BY PRECIPITATION AND RE-DISSOLUTION

(71) Applicants: North Carolina State University, Raleigh, NC (US); WAYNE STATE UNIVERSITY, Detroit, MI (US)

(72) Inventors: Thomas THEIS, Raleigh, NC (US); Patrick TOMHON, Raleigh, NC (US); Eduard Y. CHEKMENEV, Raleigh, MI (US)

(21) Appl. No.: 19/101,353

(22) PCT Filed: Aug. 21, 2023
(86) PCT No.: PCT/US2023/072533

§ 371 Date: Feb. 5, 2025

Related U.S. Application Data
(60) Provisional application No. 63/373,097, filed on Aug. 22, 2022.

(51) Int. Cl. A61K 49/18; G01R 33/28

(52) U.S. Cl. CPC A61K 49/1896; G01R 33/282

(57) ABSTRACT

In one aspect, the disclosure relates to precipitated hyperpolarized substrates, methods of making the same, contrast agents comprising the same, and methods of diagnosing and/or monitoring the progress of a disease using the same. In one aspect, the method comprises contacting a solution containing a first solvent and a hyperpolarized substrate with a non-polar organic solvent. In a further aspect, the precipitated hyperpolarized substrate can be separated from the first solvent, the non-polar organic solvent, or any combination thereof by filtration. In still another aspect, the method further includes redissolving the precipitated hyperpolarized substrate in a biocompatible solvent such as, for example, water or a physiologically-acceptable buffer. In any of these aspects, hyperpolarization of the substrate can be accomplished using Signal Amplification by Reversible Exchange (SABRE).

### CROSS-REFERENCE TO RELATED APPLICATIONS

This application claims the benefit of U.S. Provisional Application No. 63/373,097, filed on Aug. 22, 2023, which is incorporated herein by reference in its entirety.

### STATEMENT REGARDING FEDERALLY SPONSORED RESEARCH OR DEVELOPMENT

This invention was made with government support under grant number EB029829 awarded by the National Institutes of Health. The government has certain rights in the invention.

### BACKGROUND

Anatomical MRI has become an indispensable tool for medical diagnostics, but low sensitivity limits its use to imaging the most abundant substances in vivo: mostly protons of water and lipids. The low detection sensitivity of magnetic resonance (MR) is caused by the low degree of nuclear spin alignment (also termed polarization) which increases with the applied static magnetic field of the MRI system. However, even at 7 Tesla—the strongest magnetic field used for medical diagnostics in the US—the nuclear spin polarization (P) of protons is approximately 2×10⁻⁵ or 2×10⁻³%. The MRI signal is directly proportional to P, ultimately meaning that ≈99.998% of the nuclei are not detected by conventional MRI measurements; for ¹³C, the polarization is even four times lower, because of the lower gyromagnetic ratio of this nucleus. Nuclear hyperpolarization techniques can provide a transient MR signal enhancement with P of the order of unity. This massive signal gain by 4-6 orders of magnitude has enabled unprecedented imaging of low-concentrated biomolecules that are administered as hyperpolarized (HP) contrast agents. Imaging of metabolic conversion becomes possible because the administered agent and metabolic downstream products have different resonance frequencies (chemical shifts). Hence, fate of the metabolites can be mapped in vivo over the lifetime of the hyperpolarization (often few minutes for ¹³C), which is sufficiently long to detect fast metabolic processes like glycolysis and the “Warburg effect”.

HP [1-¹³C]pyruvate has emerged as the leading contrast agent due to its central role in cellular energy pathways and fast cellular uptake. Metabolic readout of HP [1-¹³C]pyruvate has been shown useful for monitoring response to treatment and grading cancer. HP [1-¹³C]pyruvate is now under evaluation in over 30 clinical trials. These promising results have been obtained using the dissolution Dynamic Nuclear Polarization (d-DNP) hyperpolarization technique. Despite the great success of d-DNP, this method still suffers from high instrumentation cost (>$2M), and complexity (cryogenic operation of superconductive magnet) as well as low throughput (approximately one polarized sample per hour using the most recent instrumentation). Impressive methodological and technical advances are being made for the technique, however, more efficient and cost-effective approaches are direly needed in order to make HP [1-¹³C]pyruvate readily accessible to the biomedical community and, ultimately, into routine clinics.

The dissolution Dynamic Nuclear Polarization (d-DNP) method has enabled revolutionary real-time MRI of metabolism without ionizing radiation using hyperpolarized [1-¹³C]pyruvate. This contrast agent has great potential, e.g., for diagnosis, prognosis, and therapy monitoring of cancer patients and is currently being evaluated in over 30 clinical trials. However, current dDNP-techniques are expensive and suffer from long hyperpolarization times posing a substantial translational roadblock for routine clinical use.

Two fast approaches have been introduced for producing [1-¹³C]pyruvate using the vast spin order readily provided by parahydrogen (p-H₂) —normal hydrogen gas enriched in the nuclear spin singlet state. Parahydrogen Induced Polarization (PHIP) can be used to hydrogenate the unsaturated side arm of esterified [1-¹³C]pyruvate. Alternatively, the non-hydrogenative Signal Amplification by Reversible Exchange (SABRE) approach is based on the simultaneous, temporary binding of the to-be-polarized substrate and p-H₂ to an Ir-IMes hexacoordinate complex and polarization transfer between the two reactants at the catalyst. SABRE in SHield Enables Alignment Transfer to Heteronuclei (SABRE-SHEATH) by conducting the exchange reaction at sufficiently low field magnetic field (typically hundreds of nanotesla). At these fields, polarization is spontaneously transferred within the exchange complex from p-H₂-derived hydride protons to 1-¹³C nucleus of the pyruvate ligand. When the transient complex is dissociated, free HP [1-¹³C]pyruvate can be created in solution state in 1 minute or less. P_{13C} of order unity has been demonstrated on the catalyst-bound [1-¹³C]pyruvate and P_{13C} 13% in the free state at relatively high (30 mM) pyruvate concentration. Rapid increase of the temperature (referred to as temperature cycling) has been shown to effectively release the pyruvate from the catalyst to obtain highly polarized solutions of [1-¹³C]pyruvate in methanol-d₄.

Despite the impressive pyruvate polarizations achieved using the SABRE route, the approach has been limited to preparing pyruvate solution in deuterated methanol in the presence of a few-millimolar iridium-based catalyst. This HP formulation is hardly suitable for direct biomedical use due to toxicity of both solvent and catalyst. A number of previous approaches have been demonstrated to capture Ir-IMes catalyst, but have not been applied to production of HP [1-¹³C]pyruvate. Moreover, pyruvate reconstitution from toxic methanol remains an unaddressed translational challenge.

Despite advances in hyperpolarization research, there is still a scarcity of methods for preparation of hyperpolarized contrast agents that are inexpensive to prepare and that can safely and effectively be administered to subjects in a clinical setting and also be used for real-time biomedical applications. These needs and other needs are satisfied by the present disclosure.

### SUMMARY

In accordance with the purpose(s) of the present disclosure, as embodied and broadly described herein, the disclosure, in one aspect, relates to precipitated hyperpolarized substrates, methods of making the same, contrast agents comprising the same, and methods of diagnosing and/or monitoring the progress of a disease using the same. In one aspect, the method comprises contacting a solution containing a first solvent and a hyperpolarized substrate with a non-polar organic solvent. In a further aspect, the precipitated hyperpolarized substrate can be separated from the first solvent, the non-polar organic solvent, or any combination thereof by filtration. In still another aspect, the method further includes redissolving the precipitated hyperpolarized substrate in a biocompatible solvent such as, for example, water or a physiologically-acceptable buffer. In any of these aspects, hyperpolarization of the substrate can be accomplished using Signal Amplification by Reversible Exchange (SABRE).

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows graphical representation of the described process for precipitation, filtration, and recovery of hyperpolarized substrates according to one embodiment of the present disclosure.

FIG. 2 shows recovery of filtered pyruvate with an aqueous flush after precipitation with chloroform from a methanol solution according to several embodiments of the present disclosure.

FIGS. 3A-3B show SABRE-SHEATH hyperpolarization of [1-¹³C]pyruvate: FIG. 3A: ¹³C NMR spectroscopy of HP [1-¹³C]pyruvate; note the three HP resonances corresponding to the species described in FIG. 3B. FIG. 3B: Schematic of simultaneous p-H₂ and [1-¹³C]pyruvate binding to the Ir-based catalyst in two different forms as described by Duckett et al.: 3a and 3b; note pyruvate binding to 3a is irreversible on the experimental time scale; thus, only 3b is SABRE-active resulting in build-up of free HP [1-¹³C]pyruvate over time in a sub-microtesla magnetic field, in FIG. 4A. [IrCI(COD)(IMes)] pre-catalyst was used (IMes=1,3-bis(2,4,6-trimethylphenyl)imidazol-2-ylidene; COD=cyclooctadiene)]).

FIGS. 4A-4I show SABRE polarization dynamics. The dynamics were analyzed in three different solvents, namely CH₃OH (circles), CD₃OD (diamonds), and a mixture of 90% C₂H₅OH and 10% H₂O (squares) using concentrations of [1-¹³C]pyruvate, SABRE catalyst, and DMSO of 30 mM (6 mM for C₂H₅OH+10% H₂O), 6 mM, and 40 mM, respectively. The SABRE-SHEATH hyperpolarization of [1-¹³C]pyruvate was investigated as a function of the magnetic field B₀ FIG. 4A, reaction temperature T FIG. 4B, time for polarization build-up at 0.4 μT FIG. 4C with corresponding extracted values of Pₘₐₓ FIG. 4D, and build-up rate FIG. 4E, T₁ relaxation of the ¹³C hyperpolarization monitored at 0.4 μT FIG. 4F and corresponding bar chart of extracted T₁ values FIG. 4G, and T₁ relaxation of the ¹³C hyperpolarization monitored at 1.4 T FIG. 4H and corresponding bar chart of extracted T₁ values FIG. 4I. The substantially reduced ¹³C T₁ of ethanol samples are likely due to a higher catalyst: substrate ratio (1:1 vs. 1:5 for methanol samples).

FIGS. 5A-5B show (FIG. 5A) Schematic of SABRE-SHEATH setup. Pressurized p-H₂ (8 bar total pressure using 100-psi overpressure regulated by a safety valve) was guided through the sample held in a 5 mm NMR tube at a flow rate of 75 scc/m using a digital mass flow controller (MFC). For the mixing step in the Re-D SABRE-SHEATH experiments, the same bubbling setup was used but the MFC was turned off and bypassed using a needle valve adjusting the flow to ≈5-10 scc/m (due to MFC instability at low scc/m rate). For hyperpolarization, the sample containing pyruvate and catalyst in CH₃OH was placed in a cold-water bath held inside a mu-metal shielded electromagnet adjusting the magnetic field to ≈0.4 μT. HP ¹³C signals were detected after transferring the samples into a benchtop 1.4 T NMR system next to the SABRE-SHEATH setup. (FIG. 5B) Schematic of additional purification procedure steps employed for Re-D SABRE after the sample removal from the shield and before the ¹³C NMR signal detection: HP [1-¹³C]pyruvate was precipitated from the organic phase by adding ethyl acetate (EtAc), redissolved in water, followed by the phase separation.

FIGS. 6A-6E show (FIG. 6A) a schematic of the procedure for HP [1-¹³C]pyruvate precipitation and Re-D SABRE at different magnetic fields. All data was acquired at 1.4 T approximately starting 10 s (FIGS. 6B, 6D) or 16 s (FIGS. 6C, 6E) after hyperpolarization, and either the precipitated samples were moved into the 1.4 T magnet and detected directly (to detect pyruvate dissolved in the supernatant, FIGS. 6B, 6D), or water was added, and samples were agitated at the respective precipitation field before moving the sample to the detection 1.4 T field (FIGS. 6C, 6E). In (FIG. 6D), the data represented by squares was measured after transfer and re-dissolution at 1.4 T. (FIG. 6B) Dependence of P_{13C} on the magnetic field value employed during HP [1-¹³C]pyruvate precipitation—note the precipitate was not dissolved, and ¹³C signal is measured from residual dissolved [1-¹³C]pyruvate in organic solvent; (FIG. 6C) Dependence of P_{13C} on the magnetic field value employed during HP [1-¹³C]pyruvate precipitation and re-dissolution-note the precipitate was dissolved, and ¹³C signal is now measured from both the residual dissolved [1-¹³C]pyruvate that remained dissolved in organic solvent and from the redissolved precipitate (FIG. 6D): T₁ decay of ¹³C HP signal measured from the residual dissolved [1-¹³C]pyruvate in organic solvent (without precipitate dissolution): T₁ decay of ¹³C HP signal measured from both the residual dissolved [1-¹³C]pyruvate in organic solvent and redissolved HP [1-¹³C]pyruvate precipitate (data was obtained from the aqueous phase with precipitate dissolution). All data in (FIG. 6D) were obtained from single point acquisitions and separate runs; (FIG. 6E) ¹³C T₁ decay of HP [1-¹³C]pyruvate after re-dissolution SABRE procedure in aqueous media (H₂O and D₂O respectively used for the re-dissolution procedure).

FIGS. 7A-7B show maximum P_{13C} levels and sample analysis of HP [1-¹³C]pyruvate in the precipitated and Re-D SABRE samples. (FIG. 7A) Sankey diagram of the procedure showing fractions of the used substances and solvents during the purification process in in the final phase-separated samples for the example experiment shown in (FIG. 7B). (FIG. 7B) ¹³C signals of a thermally-polarized reference (neat [1-¹³C]acetic acid, P_{13C}≈0.00012%, c≈17.5 M ¹³C) and HP [1-¹³C]pyruvate extracted into H₂O, P_{13C}=9±1%, c=5.8±0.8 mM, respectively. Note that the pyruvate sample was diluted by a factor of ≈3.35 compared to the initial HP sample of 30 mM pyruvate in methanol to fill the sensitive detection volume of the benchtop NMR, i.e., 65±7% of the original HP pyruvate was transferred into the H₂O.

FIG. 8 shows SABRE polarization dynamics for 60 mM HP [1-¹³C]pyruvate in CH₃OH. SABRE catalyst, and DMSO were added in concentration of 6 mM, and 40 mM, respectively. The SABRE-SHEATH hyperpolarization of [1-¹³C]pyruvate at 300 nT was investigated as function of the reaction temperature T (upper left), and time for polarization build-up (upper right). The relaxation of the ¹³C hyperpolarization was monitored at 0.30 μT (lower left) and at 1.4 T (lower right). In the three latter, mono-exponential curves were fitted to the data and suggested a polarization build-up time constant of T_{b}=(22.0±0.7) s, and T₁ relaxation times of (34.6±1.1) s and (59.3±0.5) s at 300 nT and 1.4 T, respectively. Optimal reaction temperature was found at ≈12° C.

FIGS. 9A-9C show schematics of the precipitation and purification approaches utilized in this study. After precipitating the pyruvate from CH₃OH by solvent addition, different filtering (FIGS. 9A-9B) and phase separation (FIG. 9C) approaches were tested. While the filtration approaches in FIGS. 9A-9B achieved low concentrations of residual solvents and catalyst (Tables 3 and 4, respectively), a successful implementation will require fast purification at high magnetic field. FIG. 9C is showing the phase separation purification approach used herein.

FIGS. 10A-10C show commercial filter efficiency. Residual concentration after filtration of (FIG. 10A) precipitating solvent (acetone or EtAc), (FIG. 10B) hyperpolarization solvent (CH₃OH), and (FIG. 10C) effective retained pyruvate. Data for pyruvate from both the supernatant and D₂O fractions are presented. Note that for the supernatant solution, only the 1 to 9 dilution fraction was analyzed.

FIG. 11 shows sodium pyruvate dissolved at different concentration (2, 4, 8, 20, 30, 40, 60, or 80 mM) in 500 μL methanol-OH and then added into 4500 μL of another “precipitating” solvent (acetone (1), ethyl acetate (2), or 1-propanol (3)). Photos were taken approximately 30 min after mixing the solvents and right after shaking the samples. The row below the photographs shows a 50-fold magnification of each solution with a check mark or cross indicating if precipitation was apparent or not, respectively. Note that the indicated concentrations refer to the initial pyruvate concentration in methanol, and that concentrations in the supernatant were 10-fold lower.

FIG. 12 shows 30 mM pyruvate in 500 μL CH₃OH crushed in different amounts (2, 4.5, or 9.5 mL) of acetone (left) or ethyl acetate (right).

FIG. 13 shows example spectra of high-resolution 1H NMR analysis of the supernatant and D2O samples collected from the cotton filtering. Both spectra show 1H signals from ethyl acetate (at ≈1.2 ppm, ≈2 ppm, and ≈4 ppm), methanol (≈3.3 ppm), and water (≈4.7 ppm, in case of supernatant an impurify of the used solvents). Pyruvate was detected at ≈2.2 ppm only in the D₂O extraction. Solvent concentrations in Table 3 were calculated via comparison of the NMR signal integrals with respect to the ethyl acetate-CH₃ peak at ≈2 ppm, which was calibrated to the signal of an external standard. The pyruvate concentration was quantified similarly using a second sample of 30 mM pyruvate in CD₃OD. Note that the samples were diluted prior to NMR quantification. The dilution was taken into account when computing the concentrations of individual species.

FIGS. 14A-14B show ethyl acetate precipitated sample of 30 mM pyruvate in methanol before and after H₂O addition. The microcrystalline phase is apparent before (FIG. 14A) but not after (FIG. 14B) H₂O was added and mixed by bubbling parahydrogen through the sample for 2 s. Instantaneously after the sample is left at rest the organic and aqueous phases separate with water being the denser solution settling at the bottom of the NMR tube (FIG. 14B).

FIG. 15 shows phase separation samples after 12 h exposure to ambient atmosphere. The catalyst turns reddish and clearly indicates that most of the catalyst stays in the organic phase (top of each sample) whereas the aqueous phase remains transparent. Note that some tubes in the middle are either empty or have different contents.

FIG. 16 shows example spectra of ¹H high-resolution NMR analysis of the supernatant and D₂O samples sample collected using the optimized phase-separation method (¹³C hyperpolarization results are provided in FIGS. 7A-7B). ¹H high-resolution NMR spectrum of the D₂O sample collected using the optimized phase-separation method (¹³C hyperpolarization results are provided in FIGS. 7A-7B). 1H signals from ethyl acetate (at ≈1.2 ppm, ≈2 ppm, and =4 ppm), methanol (≈3.3 ppm), and water (≈4.7 ppm) are seen. Pyruvate was detected at ≈2.2 ppm in the D₂O extraction. Solvent concentrations were calculated via comparison of the NMR signal integrals with respect to the ethyl acetate-CH₃ peak at ≈2 ppm, which was calibrated to the signal of an external standard (using 1.4 T NMR spectrometer). Pyruvate concentration was quantified using from this spectrum via peak integration and the prior knowledge (from 1.4 T data) of the solvent peak concentrations. Note that the samples were diluted prior to NMR quantification. The dilution was taken into account when computing the concentrations of individual species yielding the concentration of 5.8 mM used for ¹³C polarization quantification. Note that the volume of the starting sample (0.100 mL) was effectively expanded to 0.335 mL—taking this fact into account (and 30 mM starting pyruvate concentration), it was concluded that 65% of original pyruvate content was extracted in the aqueous phase.

FIG. 17 shows optimization of the solution mixing duration using HP [1-¹³C]pyruvate in CH₃OH. On the left panel the level of ¹³C polarization is displayed as a function of how long the mixing of the precipitated state (organic phase) with the parahydrogen catheter was applied (N=2). The rapid decrease of signal (left) was attributed to precipitation from the liquid organic phase (only solution state is detected). Mono-exponential fitting suggested that 38±11% of the 30 mM pyruvate in 100 μL methanol stayed in the solution 2 seconds after adding and mixing 400 μL ethyl acetate. On the right panel, the level of ¹³C polarization is displayed as a function of how long the mixing of the dissolved state (aqueous phase) with the parahydrogen catheter was applied (N=1). Although, this data is ambiguous, the best re-dissolution was achieved at 4 s mixing (total of 6 s including 1 s of H₂O addition and 1 s to phase separate the sample), which was used in the re-dissolution studies described herein as a trade-off of proper sample mixing and T₁ relaxation.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein is a technique for preparation of biologically compatible solutions for use as in vivo contrast agents for metabolic imaging. In one aspect, SABRE (Signal Amplification by Reversible Exchange) is a hyperpolarization technique that enables rapid preparation of solutions of endogenous metabolites with inexpensive benchtop instrumentation. In another aspect, however, SABRE solutions are currently most effectively prepared in methanol or other toxic solvents due to the role these solvent molecules can play in modulating chemical exchange. In one aspect, for future pre-clinical and clinical applications, it will be necessary to transfer the hyperpolarized molecule (e.g. pyruvate, metronidazole) into a solution compatible with injection into a live patient without harm. In a still further aspect, the catalyst used in SABRE hyperpolarization should be removed before injection. Disclosed herein is a method for transferring a SABRE hyperpolarized molecule in one solvent into a clean solution for injection into a biological system.

In one aspect, target molecules hyperpolarized with SABRE are commonly soluble in methanol and other alcohol-based solutions and insoluble in non-polar solvents due to the molecule polarities associated with biological systems. In a further aspect, by adding a large fraction of a non-polar solvent into an alcohol-based solution containing a target molecule the target molecule can be precipitated from the solution. In still another aspect, the target molecule can then be easily and rapidly filtered, washed, and redissolved in a clean biologically compatible solution for injection. In one aspect, this process requires precise magnetic field control through each of the steps in the process. In an aspect, redissolution of the target molecule in a clean solvent system results in not only a compatible solvent for injection, but also removal of the potentially toxic SABRE catalyst. In one aspect, and without wishing to be bound by theory, hyperpolarization of pyruvate and other target substrates in ethanol may not work well due to limited solubility of the target substrates in ethanol.

Also disclosed herein is a method for preparing a precipitated hyperpolarized substrate, the method including contacting a solution containing a first solvent and a hyperpolarized substrate with a non-polar organic solvent. In another aspect, the non-polar organic solvent can include an unpolarized but otherwise identical substrate in a concentration of from about 1 μM to about 100 mM, or of about 1, 50, 100, 250, 500, or 750 μM or about 1, 5, 10, 25, 50, 75, or about 100 mM, or a combination of any of the foregoing values, or a range encompassing any of the foregoing values. In some aspects, the presence of the substrate in an unpolarized form (or a thermally polarized form) in the non-polar organic solvent at low concentration may aid in rapid precipitation or crystallization of the hyperpolarized substrate.

In another aspect, in the disclosed method, the precipitated hyperpolarized substrate can be separated from the first solvent, the non-polar organic solvent, or any combination thereof. In a further aspect, separating can be accomplished by filtration. In one aspect, filtration can be carried out using a C18 silica filter, a C9 silica filter, a micro-scale filter, a cellulose acetate filter, a cotton filter, or any combination thereof.

In one aspect, the method further includes redissolving the precipitated hyperpolarized substrate in a biocompatible solvent such as, for example, water or a physiologically-acceptable buffer. In another aspect, the physiologically-acceptable buffer can be saline, phosphate buffered saline, sodium or potassium phosphate buffer, bicarbonate buffer, 2-(N-morpholino) ethanesulfonic acid (MES), bis-(2-hydroxyethyl) amino-tris(hydroxymethyl) methane (Bis-Tris), N-(2-acetamido)iminodiacetic acid (ADA), N-(carbamoylmethyl)-2-aminoethane sulfonic acid (ACES), 2-[4-(2-sulfoethyl)piperazin-1-yl]ethanesulfonic acid (PIPES), 3-morpholino-2-hydroxypropanesulfonic acid (MOPSO), N,N-bis(2-hydroxyethyl)taurine (BES), 3-(N-morpholino) propanesulfonic acid (MOPS), 2-[4-(2-hydroxyethyl)piperazin-1-yl]ethanesulfonic acid (HEPES), N-[tris(hydroxymethyl)methyl]2-aminoethanesulfonic acid (TES), 2-amino-2-(hydroxymethyl)-1,3-propanediol (Tris), N-(hydroxyethyl) piperazine-N′-2-hydroxypropanesulfonic acid (HEPPSO), N-[tris(hydroxymethyl)methyl]glycine (Tricine), N, N-bis(hydroxyethyl)glycine (Bicine), N-[tris(hydroxymethyl)methyl]-3-aminopropanesulfonic acid (TAPS), boric acid buffer, N-cyclohexyltaurine (CHES), or any combination thereof.

In another aspect, the non-polar organic solvent can be selected from chloroform, diethyl ether, ethyl acetate, acetone, ethanol, acetic acid, dichloromethane, toluene, xylene, a perfluoropolyether solvent, a hydrofluoroether solvent, a methylsiloxane, a C4-C10 alkane or cycloalkane, or any combination thereof.

Further perfluoropolyether solvents useful herein include, but are not limited to, GALDEN® perfluoropolyether fluorinated fluids from Solvay, S.A. (Brussels, Belgium) such as, for example, GALDEN® HT55, GALDEN® HT80, GALDEN® HT110, GALDEN® HT135, GALDEN® HT170, GALDEN® HT200, GALDEN® HT230, GALDEN® HT270, or any combination thereof. Further hydrofluoroether solvents useful herein include, but are not limited to, NOVEC® solvents from 3M Company (St. Paul, MN, US) such as, for example, NOVEC® 649 or 1230. Further methylsiloxane solvents useful herein include, but are not limited to, hexamethyldisiloxane, octamethyltrisiloxane, decamethyltetraisiloxane, decamethylpentasiloxane, or any combination thereof. Still further non-polar organic solvents useful herein include 1,3-bis(trifluoromethyl)benzene, perfluoro hexane, AE-3000, 1H,1H,7H-dodecafluoro-1-heptanol, HFE-7100, HFE-7200, HFE-7300, 1-bromoperfluorooctane, HFE-7000, hexadecane, octadecane, PAO6, and combinations thereof.

In still another aspect, the precipitated hyperpolarized substrate can be a drug or metabolite such as, for example, pyruvate, oxaloglutarate, oxaloacetate, phenyl pyruvate, 2-oxo-butyrate, 2-oxoglutarate, urea, 2,3-diketogluatarate, 2-oxo-adipate, acetonitrile, benzonitrile, α-cyano-4-hydroxycinnamic acid (CHCA), alectinib, metronidazole, dichloropyridazine, nicotinamide, imidazole, adenine, diphenyldiazene, diazirine, or any combination thereof. In a further aspect, the precipitated hyperpolarized substrate can be hyperpolarized on at least one nucleus selected from ¹H, ¹⁵N, ¹³C, or any combination thereof.

In any of these aspects, prior to performing the method, the hyperpolarized substrate can be produced using signal amplification by reversible exchange (SABRE). In one aspect, SABRE can be performed at a temperature of from about −10° C. to about 100° C., in a magnetic field of from about 0 μT to about 100 mT. In a further aspect, SABRE can be performed at about −10, 0, 10, 20, 30, 40, 50, 60, 70, 80, 90, or about 100° C., or a combination of any of the foregoing values, or a range encompassing any of the foregoing values. In another aspect, the magnetic field can have a strength of about 0, 50, 100, 250, 500, or 750 μT or about 1, 5, 10, 25, 50, 75, or about 100 mT, or a combination of any of the foregoing values, or a range encompassing any of the foregoing values. In one aspect, controlling the magnetic field during SABRE maximizes hyperpolarization. In another aspect, controlling temperature during hyperpolarization enables a greater degree of hyperpolarization.

In another aspect, contacting the solution with the first solvent and the hyperpolarized substrate with the non-polar organic solvent can be performed at a temperature of from about −20° C. to about 50° C., in a magnetic field of from about 0 μT to about 10 T. In a further aspect, contacting can be carried out at about −20, −10, 0, 10, 20, 30, 40, or about 50° C., or a combination of any of the foregoing values, or a range encompassing any of the foregoing values. In another aspect, the magnetic field can be about 0, 50, 100, 250, 500, or 750 μT or about 1, 5, 10, 25, 50, 75, 100, 250, 500, or 750 mT, or about 1, 5, or about 10 T, or a combination of any of the foregoing values, or a range encompassing any of the foregoing values. In another aspect, controlling the magnetic field and temperature during precipitation minimize relaxation losses.

In still another aspect, filtration can be performed at a temperature of from about −20° C. to about 50° C., in a magnetic field of from about 0 μT to about 10 T. In a further aspect, filtering can be carried out at about −20, −10, 0, 10, 20, 30, 40, or about 50° C., or a combination of any of the foregoing values, or a range encompassing any of the foregoing values. In another aspect, the magnetic field can be about 0, 50, 100, 250, 500, or 750 μT or about 1, 5, 10, 25, 50, 75, 100, 250, 500, or 750 mT, or about 1, 5, or about 10 T, or a combination of any of the foregoing values, or a range encompassing any of the foregoing values. In another aspect, controlling the magnetic field and temperature during filtration minimize relaxation losses.

In yet another aspect, redissolving the precipitated hyperpolarized substrate can be performed at a temperature of from about 0° C. to about 100° C., in a magnetic field of from about 0 μT to about 10 T. In a further aspect, redissolving can be conducted at about 0, 10, 20, 30, 40, 50, 60, 70, 80, 90, or about 100° C., or a combination of any of the foregoing values, or a range encompassing any of the foregoing values. In still another aspect, the magnetic field can be about 0, 50, 100, 250, 500, or 750 μT or about 1, 5, 10, 25, 50, 75, 100, 250, 500, or 750 mT, or about 1, 5, or about 10 T. In another aspect, controlling the magnetic field and temperature during redissolution minimize relaxation losses.

In any of these aspects, magnetic field can be controlled by a radio frequency coil, a shielding mechanism, an electromagnet, a solenoid powered with direct current, a permanent magnet or permanent magnet array, a superconducting magnet, or any combination thereof. In another aspect, the method can be automated. In one aspect, and without wishing to be bound by theory, automation of the disclosed method allows a reproducible and fast process that minimizes depolarization losses to the T₁ relaxation of the hyperpolarized state.

In one aspect, the means for controlling the magnetic field at any step in the disclosed process can be a radio frequency coil. In another aspect, the means for controlling the magnetic field can be a shielding mechanism to reduce the influence of Earth's magnetic field. Further in this aspect, such a shielding mechanism can allow access to a micro Tesla magnetic field. In still another aspect, the means for controlling the magnetic field can be a solenoid powered with a direct current to establish the desired magnetic field. In yet another aspect, the means for controlling the magnetic field can be a permanent magnet array. In another aspect, the means for controlling the magnetic field can be a superconducting magnet. In some aspects, two or more of these means for controlling the magnetic field can be used simultaneously or sequentially. In one aspect, the ideal magnetic field can be selected based on the details of the chemical system and the spin physics of the hyperpolarization transfer process from parahydrogen to other nuclei. In one aspect, the shielding mechanism is or incorporates mu-metal. In a further aspect, mu-metal is a nickel-iron soft ferromagnetic alloy with very high permeability useful in shielding applications. A non-limiting example of a mu-metal composition can be 77% nickel, 16% iron, 5% copper, and 2% chromium or molybdenum. A second non-limiting example of a mu-metal composition can be 80% nickel, 5% molybdenum, small amounts of silicon and/or other elements, and the remaining 12 to 15% iron. Other compositions are also envisioned. In some aspects, the shielding mechanism can be a commercial product such as, for example, a Twinleaf MS-1L compact magnetic shield (Twinleaf LLC).

In some aspects, a current can be applied to the means for controlling the magnetic field in order to generate the magnetic field. In a further aspect, the current applied to the means for controlling the magnetic field has a frequency of from about 0 Hz to about 300 GHz, or of from about 0 Hz to about 1 GHz. In another aspect, the current has a frequency of about 100 kHz, 200 kHz, 300 kHz, 400 kHz, 500 kHz, 600 kHz, 700 kHz, 800 kHz, 900 kHz, 1 MHz, 100 MHz, 200 MHz, 300 MHz, 400 MHz, 500 MHz, 600 MHz, 700 MHz, 800 MHz, 900 MHz, 1 GHz, 100 GHz, 200 GHz, or about 300 GHz, or a combination of any of the foregoing values, or a range encompassing any of the foregoing values. In one aspect, the frequency is about 400 kHz or is about 1 GHz. In another aspect, the current is a direct current.

Also disclosed herein are precipitated hyperpolarized substrates prepared by the disclosed methods, and/or redissolved hyperpolarized substrates prepared by the disclosed methods. In another aspect, disclosed herein are biocompatible contrast agents containing the precipitated hyperpolarized substrates and/or the redissolved hyperpolarized substrates.

In another aspect, disclosed herein is a method for diagnosing a disease or monitoring progress of treatment of disease in a subject, the method including at least the steps of:
(a) administering the precipitated hyperpolarized substrate, the redissolved hyperpolarized substrate, or the contrast agent to the subject; and
(b) performing imaging on the subject,
wherein performing imaging enables visualization of the precipitated hyperpolarized substrate or redissolved hyperpolarized substrate in the subject.

In one aspect, the subject can be a mammal such as, for example, a human, mouse, rat, pig, hamster, guinea pig, sheep, dog, cat, or horse. In another aspect, the precipitated hyperpolarized substrate, redissolved hyperpolarized substrate, or contrast agent can be administered to the subject in a single injection, or can be administered to the subject continuously for a period of from about 30 seconds to about 1 hour, or for about 30 seconds or about 1, 5, 10, 15, 20, 25, 30, 45, or about 60 minutes, or a combination of any of the foregoing values, or a range encompassing any of the foregoing values.

In another aspect, the disease can be cancer, cardiovascular disease, or a metabolic disorder. In one aspect, the cancer can be prostate cancer, breast cancer, or brain cancer. In still another aspect, the metabolic disorder can be diabetes, pyruvate dehydrogenase complex deficiency, or pyruvate carboxylase deficiency.

In any of these aspects, the imaging can be magnetic resonance imaging (MRI). In one aspect, the MRI is carried out using a cryogen-cooled superconducting magnet or a cryogen-free magnet.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a catalyst,” “a non-polar organic solvent,” or “a buffer,” include, but are not limited to, mixtures or combinations of two or more such catalysts, non-polar organic solvents, or buffers, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x,’ ‘about y’, and ‘about z’ as well as the ranges of ‘less than x’, less than y’, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a polarization transfer catalyst refers to an amount that is sufficient to achieve the desired improvement in the property modulated by the formulation component, e.g. achieving the desired level of hyperpolarization. The specific level in terms of wt % in a composition required as an effective amount will depend upon a variety of factors including the amount and type of catalyst, amount and type of target molecule or substrate, amount and type of solvent, and presence and identity of any co-ligands.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

“Thermal polarization” as used herein refers to the fraction of nuclear spins that align with a magnetic field under normal conditions. This is typically a small number and can be measured in units of parts per million (ppm), even in a strong magnetic field.

By contrast, “hyperpolarization” refers to nuclear spin polarization far beyond thermal equilibrium conditions. In one aspect, hyperpolarization aligns almost all spins with the magnetic field, achieving signal enhancements of up to 10,000,000-fold when compared to thermal polarization.

“Orthohydrogen” (0-H₂) is an isomeric form of molecular hydrogen. In o-H₂, the spins of both nuclei are symmetrically aligned. In one aspect, at room temperature and thermal equilibrium, approximately 75% of an H₂ sample is in the orthohydrogen (triplet) state.

“Parahydrogen” (p-H₂) is a second isomeric form of molecular hydrogen. In p-H₂, the spins of both nuclei are anti-symmetrically aligned. In one aspect, at room temperature and thermal equilibrium, approximately 25% of an H₂ sample is in the parahydrogen (singlet) state. In a further aspect, use of parahydrogen exhibits hyperpolarized signals in NMR spectra. In one aspect, the reactor and process disclosed herein use parahydrogen to induce transfer spin in order to induce hyperpolarization in samples for NMR and MRI analysis. “Parahydrogen Induced Polarization” or “PHIP” is a hyperpolarization technique using p-H₂ as a source of spin transfer for inducing hyperpolarization. In one aspect, PHIP involves chemical reaction of p-H₂.

As used herein, a “cryogen-free magnet” can refer to a solid state magnet array or to a “dry” magnet that does not consume liquid helium or liquid nitrogen but rather uses compressed recycled helium, which can be liquefied, to cool the magnet.

“Signal amplification by reversible exchange” or “SABRE” is a technique that can increase the visibility of compounds for the purpose of NMR and MRI analysis, which in turn allows lower detection limits and shorter scan times in NMR, as well as higher contrast and higher resolution in MRI imaging. In one aspect, a metal-containing catalyst transfers spin from parahydrogen to a substrate, which can then be imaged or analyzed as appropriate.

As used herein, a “polarization transfer catalyst” is a metal containing catalyst that transiently binds both a substrate molecule and p-H₂, thereby allowing polarization to transfer from the p-H₂ to the substrate in a magnetic field. In some aspects, the metal in the polarization transfer catalyst is iridium. In another aspect, the iridium is typically coordinated with species containing aromatic rings and/or nitrogen heterocycles.

In some aspects, a “co-ligand” can be used in the disclosed methods. As used herein, “co-ligand” refers to a molecule capable of coordinating with the metal center in a polarization transfer catalyst. A co-ligand can, in some aspects, enhance polarization transfer efficiency to a target molecule, or can enhance binding efficiency of target molecules to the polarization transfer catalyst, or any combination thereof. Useful co-ligands disclosed herein include, but are not limited to, DMSO, water, and combinations thereof.

As used herein, “substrate” and “target molecule” refer to a molecule or chemical species to which polarization transfer is desired. Substrate and/or target molecules may be bound to a polarization transfer catalyst, may be free in solution, or a combination thereof.

As used herein, a “metabolite” is any substance formed by a metabolic process or necessary for a metabolic process. In this aspect, a metabolite can be a protein, peptide, nucleic acid, sugar, lipid, vitamin, or a subunit or component thereof (e.g. amino acid, nucleobase, nucleoside, nucleotide, monosaccharide, disaccharide, fatty acid, cofactor, or any combination thereof). Meanwhile, as used herein, “drug” refers to any substance that has a physiological effect when introduced to at least one tissue or organ system. In an aspect, both metabolites and drugs can include small molecules produced by animals, plants, bacteria, fungi, algae, and/or other organisms including, but not limited to, plant secondary metabolites (e.g. alkaloids, terpenoids, phenolic compounds, polyketides, non-ribosomal peptides), antibiotics, and the like. In a further aspect, drugs can additionally include synthetic and semi-synthetic compounds.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

Unless otherwise specified, pressures referred to herein are based on atmospheric pressure (i.e. one atmosphere).

### Process for Preparing Hyperpolarized Contrast Agents in Aqueous Solution

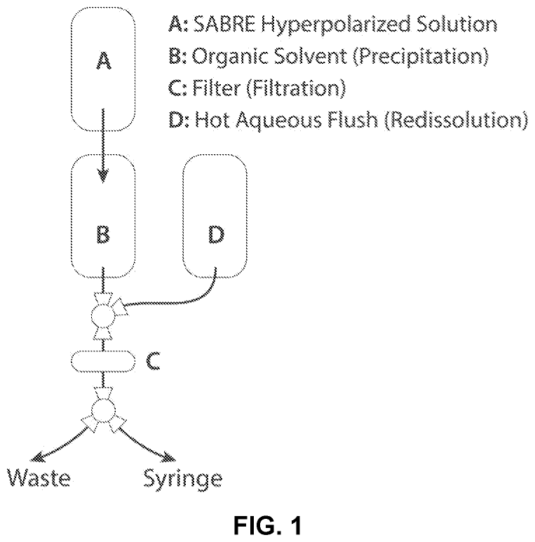

In one aspect, in the disclosed process, the hyperpolarized pyruvate is first extracted from an alcohol-based solution by precipitation with a miscible biosafe organic solvent and then extracted using a standard micron-scale filter. Disposal of the organic fraction then enables rapid recovery of the solid hyperpolarized pyruvate from the filter using a hot aqueous flush (see FIG. 1). This process is described in further detail in the Examples.

All volumes, pressures, concentrations, magnetic fields, chemicals, and other details below are given as examples for the purpose of the technical description of this technology for a generic and descriptive sense only and not for purposes of limitation.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered Aspects, which should not be confused with the claims.

Aspect 1. A method for preparing a precipitated hyperpolarized substrate, the method comprising contacting a solution comprising a first solvent and a hyperpolarized substrate with a non-polar organic solvent.

Aspect 2. The method of aspect 1, wherein the non-polar organic solvent comprises an unpolarized but otherwise identical substrate in a concentration of from about 1 μM to about 100 mM.

Aspect 3. The method of aspect 1 or 2, further comprising separating the precipitated hyperpolarized substrate from the first solvent, the non-polar organic solvent, or any combination thereof.

Aspect 4. The method of aspect 3, wherein separating is accomplished by filtration.

Aspect 5. The method of aspect 4, wherein filtration is carried out using a C18 silica filter, a C9 silica filter, a micro-scale filter, a cellulose acetate filter, a cotton filter, or any combination thereof.

Aspect 6. The method of any one of aspects 2-5, further comprising redissolving the precipitated hyperpolarized substrate in a biocompatible solvent.

Aspect 7. The method of aspect 6, wherein the biocompatible solvent comprises water or a physiologically-acceptable buffer.

Aspect 8. The method of aspect 7, wherein the physiologically-acceptable buffer comprises saline, phosphate buffered saline, sodium or potassium phosphate buffer, bicarbonate buffer, 2-(N-morpholino) ethanesulfonic acid (MES), bis-(2-hydroxyethyl) amino-tris(hydroxymethyl) methane (Bis-Tris), N-(2-acetamido)iminodiacetic acid (ADA), N-(carbamoylmethyl)-2-aminoethane sulfonic acid (ACES), 2-[4-(2-sulfoethyl) piperazin-1-yl]ethanesulfonic acid (PIPES), 3-morpholino-2-hydroxypropanesulfonic acid (MOPSO), N,N-bis(2-hydroxyethyl)taurine (BES), 3-(N-morpholino)propanesulfonic acid (MOPS), 2-[4-(2-hydroxyethyl)piperazin-1-yl]ethanesulfonic acid (HEPES), N-[tris(hydroxymethyl)methyl]2-aminoethanesulfonic acid (TES), 2-amino-2-(hydroxymethyl)-1,3-propanediol (Tris), N-(hydroxyethyl) piperazine-N′-2-hydroxypropanesulfonic acid (HEPPSO), N-[tris(hydroxymethyl)methyl]glycine (Tricine), N, N-bis(hydroxyethyl)glycine (Bicine), N-[tris(hydroxymethyl)methyl]-3-aminopropanesulfonic acid (TAPS), boric acid buffer, N-cyclohexyltaurine (CHES), or any combination thereof.

Aspect 9. The method of any one of aspects 1-8, wherein the non-polar organic solvent comprises chloroform, diethyl ether, ethyl acetate, acetone, ethanol, acetic acid, dichloromethane, toluene, xylene, a perfluoropolyether solvent, a hydrofluoroether solvent, a methylsiloxane, a C4-C10 alkane or cycloalkane, or any combination thereof.

Aspect 10. The method of any one of aspects 1-9, wherein the precipitated hyperpolarized substrate comprises a drug or a metabolite.

Aspect 11. The method of any one of aspects 1-10, wherein the precipitated hyperpolarized substrate comprises pyruvate, oxaloglutarate, oxaloacetate, phenyl pyruvate, 2-oxo-butyrate, 2-oxoglutarate, urea, 2,3-diketogluatarate, 2-oxo-adipate, acetonitrile, benzonitrile, α-cyano-4-hydroxycinnamic acid (CHCA), alectinib, metronidazole, dichloropyridazine, nicotinamide, imidazole, adenine, diphenyldiazene, diazirine, or any combination thereof.

Aspect 12. The method of any one of aspects 1-11, wherein the precipitated hyperpolarized substrate is hyperpolarized on at least one nucleus selected from 1H, 15N, 13C, or any combination thereof.

Aspect 13. The method of any one of aspects 1-12, wherein prior to performing the method, the hyperpolarized substrate is produced using signal amplification by reversible exchange (SABRE).

Aspect 14. The method of aspect 13, wherein SABRE is performed at a temperature of from about −10° C. to about 100° C.

Aspect 15. The method of aspect 13 or 14, wherein SABRE is performed in a magnetic field of from about 0 μT to about 100 mT.

Aspect 16. The method of any one of aspects 1-15, wherein contacting the solution comprising the first solvent and the hyperpolarized substrate with the non-polar organic solvent is performed at a temperature of from about −20° C. to about 50° C.

Aspect 17. The method of any one of aspects 1-16, wherein contacting the solution comprising the first solvent and the hyperpolarized substrate with the non-polar organic solvent is performed in a magnetic field of from about 0 T to about 10 T.

Aspect 18. The method of any one of aspects 4-17, wherein filtration is performed at a temperature of from about −20° C. to about 50° C.

Aspect 19. The method of any one of aspects 4-18, wherein filtration is performed in a magnetic field of from about 0 T to about 10 T.

Aspect 20. The method of any one of aspects 6-19, wherein redissolving the precipitated hyperpolarized substrate is performed at a temperature of from about 0° C. to about 100° C.

Aspect 21. The method of any one of aspects 6-20, wherein redissolving the precipitated hyperpolarized substrate is performed in a magnetic field of from about 0 T to about 10 T.

Aspect 22. The method of any one of aspects 15, 17, 19, or 21, wherein the magnetic field is controlled by a means for controlling the magnetic field selected from a radio frequency coil, a shielding mechanism, an electromagnet, a solenoid powered with direct current, a permanent magnet or permanent magnet array, a superconducting magnet, or any combination thereof.

Aspect 23. The method of aspect 22, wherein the magnetic field is generated by applying a current to the means for controlling the magnetic field.

Aspect 24. The method of aspect 23, wherein the current has a frequency of from about 0 Hz to about 300 GHz.

Aspect 25. The method of any one of aspects 1-24, wherein the method is automated.

Aspect 26. A precipitated hyperpolarized substrate prepared by the method of any one of aspects 1-25.

Aspect 27 A redissolved hyperpolarized substrate prepared by the method of any one of aspects 6-25.

Aspect 28. A biocompatible contrast agent comprising the precipitated hyperpolarized substrate of aspect 26 or the redissolved hyperpolarized substrate of aspect 27.

Aspect 29. A method for diagnosing a disease or monitoring progress of treatment of a disease in a subject, the method comprising:
(a) administering the precipitated hyperpolarized substrate of aspect 26, the redissolved hyperpolarized substrate of aspect 27, or the contrast agent of aspect 28 to the subject; and
(b) performing imaging on the subject,
wherein performing imaging enables visualization of the precipitated hyperpolarized substrate or redissolved hyperpolarized substrate in the subject.

Aspect 30. The method of aspect 29, wherein the subject is a mammal.

Aspect 31. The method of aspect 24-30 wherein the mammal is a human, mouse, rat, pig, hamster, guinea pig, sheep, dog, cat, or horse.

Aspect 32. The method of any one of aspects 29-31, wherein the precipitated hyperpolarized substrate, redissolved hyperpolarized substrate, or contrast agent is administered to the subject in a single injection.

Aspect 33. The method of any one of aspects 29-31, wherein the precipitated hyperpolarized substrate, redissolved hyperpolarized substrate, or contrast agent is administered to the subject continuously for a period of from about 30 seconds to about 1 hour.

Aspect 34. The method of any one of aspects 29-33, wherein the disease comprises cancer, cardiovascular disease, or a metabolic disorder.

Aspect 35. The method of aspect 34, wherein the cancer comprises prostate cancer, breast cancer, or brain cancer.

Aspect 36. The method of aspect 34, wherein the metabolic disorder comprises diabetes, pyruvate dehydrogenase complex deficiency, or pyruvate carboxylase deficiency.

Aspect 37. The method of any one of aspects 29-36, wherein the imaging is magnetic resonance imaging (MRI).

Aspect 38. The method of aspect 37, wherein magnetic resonance imaging is carried out using a cryogen-cooled superconducting magnet.

Aspect 39. The method of aspect 37, wherein magnetic resonance imaging is carried out using a cryogen-free magnet.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: General Process

In one aspect, 75 mM [1-13C]-pyruvate is hyperpolarized in a hyperpolarization reactor using SABRE in a temperature cycling motif at a pressure between 150-200 psi, generating ˜1 mL of a highly polarized solution of [1-13C]-pyruvate (as described in Reference [1]).

The solution is then transferred into a secondary vessel using gated back-pressure, rapidly injecting 1 mL of hyperpolarized solution into 3 mL of chloroform solution (or another, more biocompatible, non-polar solvent) containing 0 to 10 mM unlabeled pyruvate to increase crystal formation of the hyperpolarized pyruvate.

The solution is then rapidly ejected out of the secondary vessel through a 0.2-μm filter using a three-way manual or solenoid valve (or a series of two-way valves) to send the organic fraction to waste collection.

A 1-mL hot (˜70° C.) aqueous flush is then rapidly injected through the secondary container and filtered to recover the hyperpolarized pyruvate, activating the three-way solenoid valve to send this solution into a syringe for collection.

The aqueous hyperpolarized [1-13C]-pyruvate solution in the syringe can then be injected and measured in NMR and MRI experiments.

The precipitation, filtration, and recovery process above can be contained in a Halbach array (>300 mT) to minimize relaxation of the hyperpolarized 13C spins during this solution preparation process. See also FIG. 1.

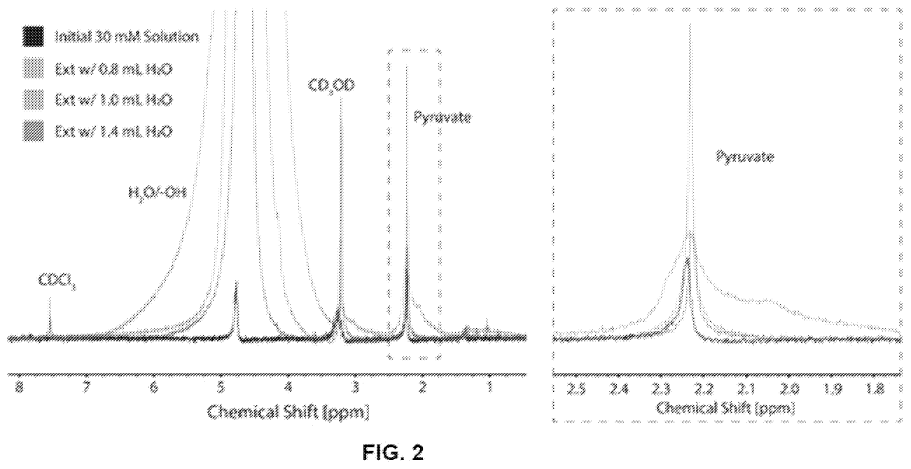

Recovery of Pyruvate in Aqueous Fractions: To verify the validity of this method with non-hyperpolarized material, where the substrate is precipitated and recovered by the method described above, FIG. 2 shows the recovery of pyruvate in aqueous fractions from an original methanol solution.

### Example 2: SABRE-SHEATH Polarization of [1-¹³C]Pyruvate

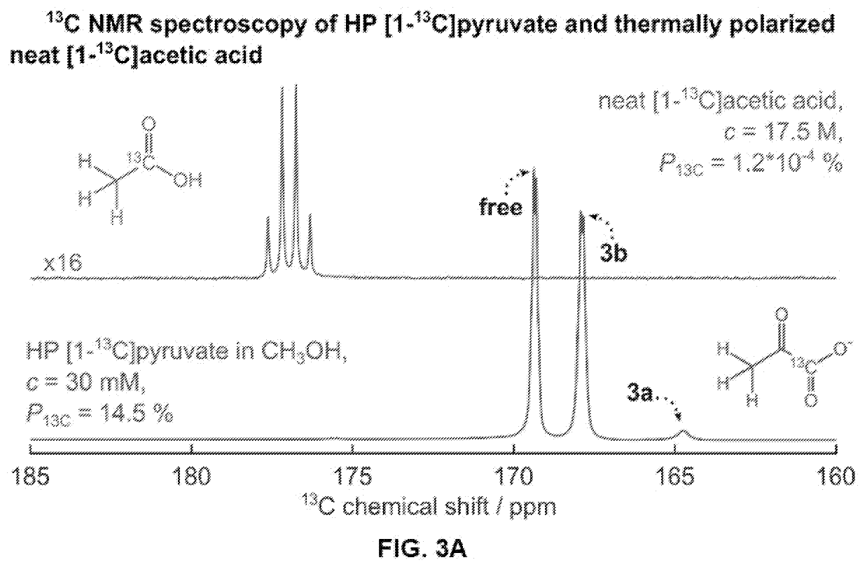

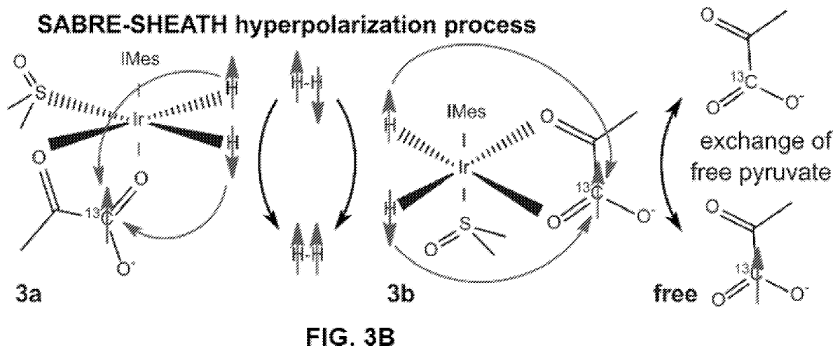

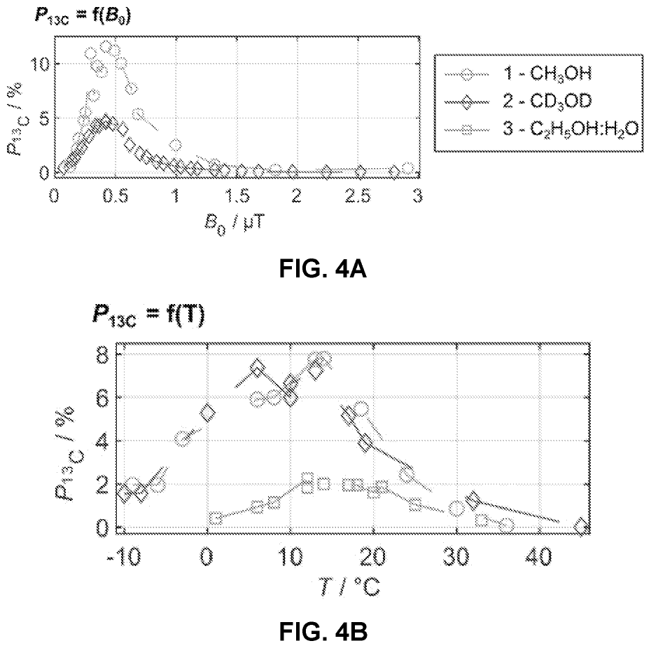

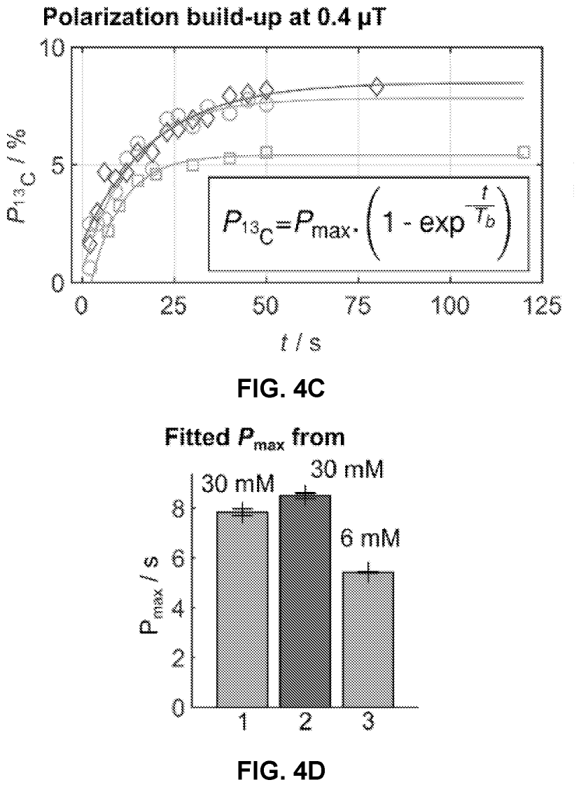

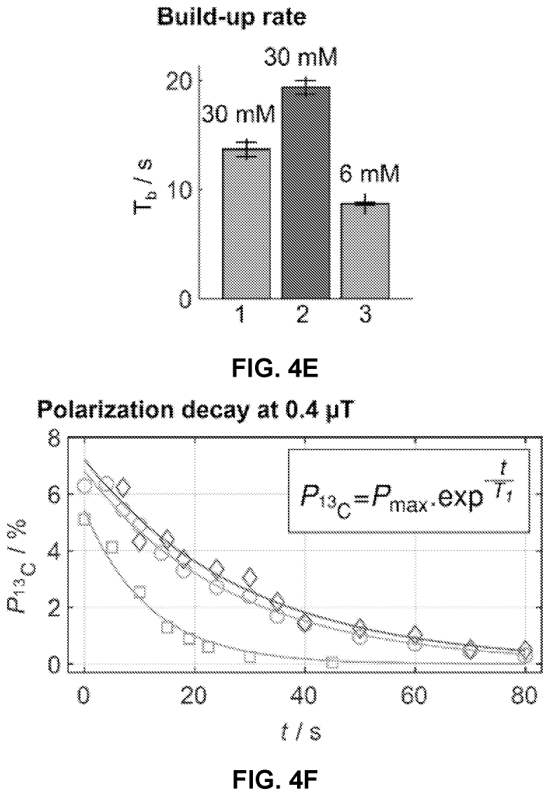

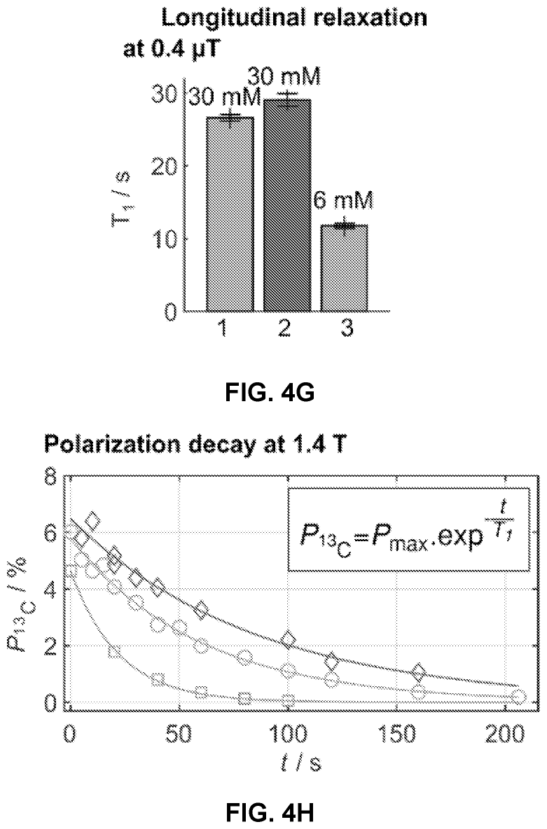

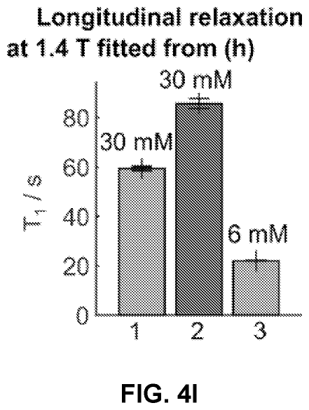

A previously reported setup and experimental protocol has been used to prepare high P_{13C} of up to ≈14.5% of 30 mM [1-¹³C]pyruvate in methanol, which was confirmed after transferring the HP samples from the polarizer into a 1.4 T benchtop ¹H/¹³C NMR system. The mean of N=19 experiments from 9 samples measured on 5 different days under the same conditions was 8±2%. FIG. 3A shows the spectrum of HP [1-¹³C]pyruvate versus the signal from a corresponding reference spectrum of thermally-polarized [1-¹³C]acetic acid (17.5 M, P_{13C}≈1.2·10⁻⁶ at B₀=1.4 T). These hyperpolarization values correspond to ¹³C enhancement ε_{13C} of ≈75,000-fold. The optimal experimental parameters are obtained via sweeping the static magnetic field (optimum transfer field B_{T}≈0.4 μT) and temperature (T_{T}≈10° C.) using a p-H₂ flow rate of 75 standard cubic centimeters per minute (scc/m), and ≈7 bar total pressure, FIGS. 4A-4B, respectively.

### Example 3: [1-¹³C]Pyruvate Polarization Dynamics in Protonated Methanol and Ethanol Solvents

CD₃OD is widely used as the SABRE-reaction-hosting solvent because it provides convenient means of attaining high-field NMR spectrometer lock. However, non-deuterated polarization media may be preferred for biomedical applications. For this reason, systematic relaxation dynamics studies were performed comparing CD₃OD, CH₃OH, and C₂H₅OH for SABRE-SHEATH [1-¹³C]pyruvate hyperpolarization, investigating relevant parameters for these solvents and for fixed concentrations of pyruvate, SABRE catalyst, and DMSO (30 mM, 6 mM, and 40 mM, respectively). However, the ethanol sample was prepared with only 6 mM pyruvate and 10% H₂O due to limited solubility. The optimal Br was similar in all investigated samples (≈0.4 μT, FIG. 4A). The highest [1-¹³C]pyruvate polarizations were observed at T_{T}=9-14° C. in both methanol samples, and for ethanol at 12-18° C. (FIG. 4B). Thus, all the experiments described below were performed using these optimized Br and Tr parameters.

It was found that the hyperpolarization was similarly effective in CD₃OD and CH₃OH. The polarization build-up (T_{b}) and decay (T₁) at 0.4 μT were similar: T_{b}=13.7±0.6 s versus 19.3±0.6 s; and T₁=26.0±0.4 s versus 29.1±0.8 s, respectively (FIGS. 4C, 4E). P_{13C} values were also similar; in one representative example of polarization build-up, P_{13C} was 7.8±0.1% vs. 8.5±0.1% for CH₃OH and CD₃OD media, respectively, FIG. 4D. The ¹³C T₁ relaxation time at high field (i.e., a clinically relevant field of 1.4 T) was significantly greater in the deuterated solvent, 86±2 s versus 59.3±0.8 s. For ethanol, 1.4-fold lower P_{13C} values were obtained in this mixture compared to CH₃OH and CD₃OD, which is rationalized by a more efficient relaxation at the SABRE-SHEATH field, FIG. 4E. Hence, despite the higher biocompatibility of ethanol, CH₃OH was selected as the solvent for further experiments due to higher sodium pyruvate solubility, longer relaxation times, and higher P_{13C} levels.

### Example 4: Precipitation of Pyruvate from Solutions

Although previous studies have demonstrated the direct SABRE polarization in biocompatible solvents such as aqueous media, the resulting polarization is markedly (by an order of magnitude or more) reduced compared to that in methanol, presumably due to lower hydrogen solubility and other compounding factors. As a result, experiments were continued with methanol as solvent for SABRE-SHEATH, and the idea of performing precipitation and purification of the HP biomolecule was investigated. In those studies the precipitation was achieved by adding an acid to the ¹³C-hyperpolarized solutions, whereas here precipitation of pyruvate is achieved by addition of a non-polar solvent. The present strategy may be advantageous for future clinical applications because it avoids the use of strong acids and bases immediately before in vivo administration, which leads to additional purification and quality-assurance steps, posing translational challenges. Systematic screening of the solvent used for precipitation was performed in order to meet the following requirements: (a) low solubility of sodium pyruvate to initiate the precipitation; (b) high solubility of the SABRE catalyst to enable its removal from the HP sample; (c) miscibility with methanol to achieve rapid mixing of solutions and pyruvate precipitation; and (d) low toxicity. The screening (see SI for details) revealed that among the options tested, ethyl acetate (EtAc) provided the best performance.

### Example 5: Re-Dissolution (Re-D) SABRE at Different Magnetic Fields

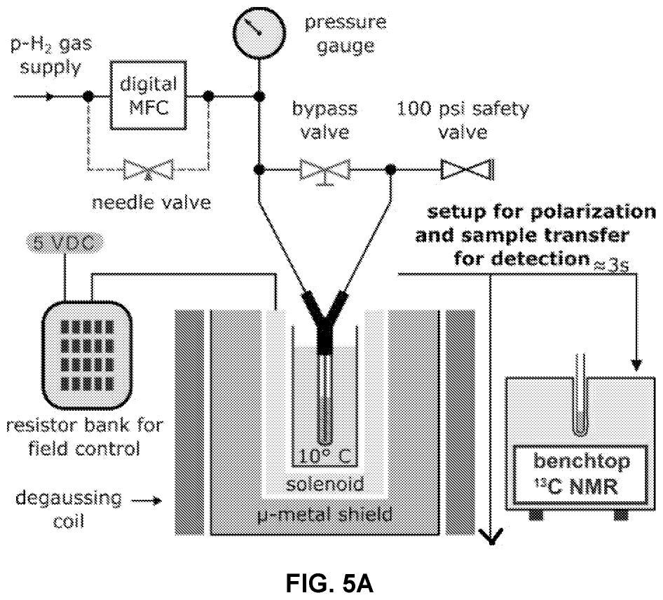

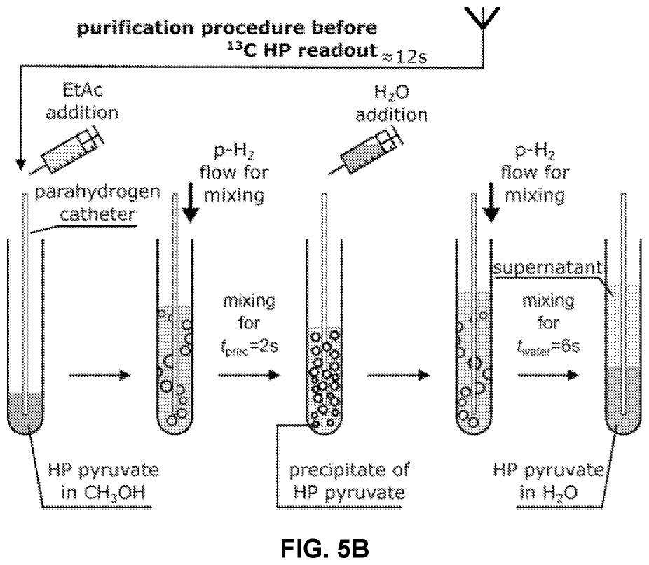

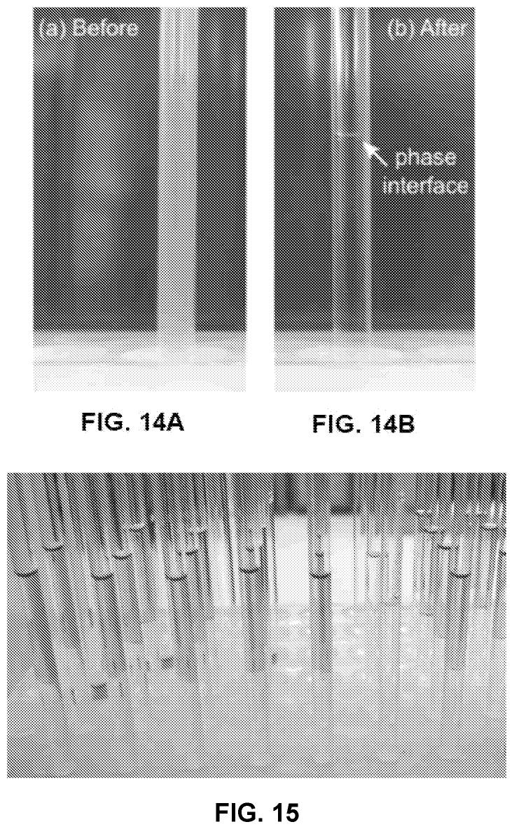

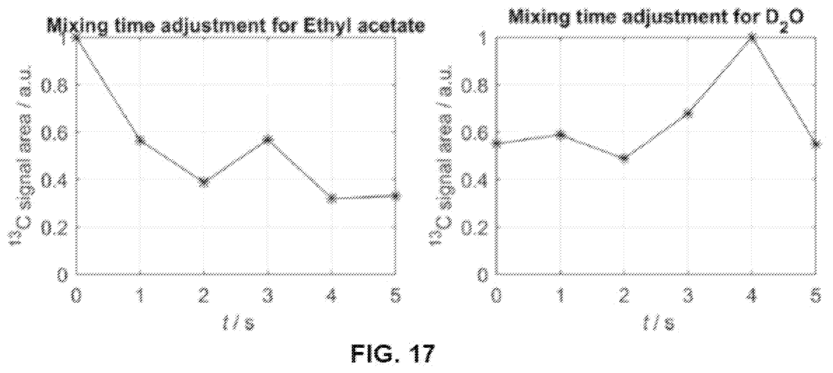

To investigate the precipitation and re-dissolution of HP [1-¹³C]pyruvate, a phase separation approach was applied, FIGS. 5A-5B. To this end, the fact that water and EtAc are immiscible was exploited to modify the setup and procedure accordingly. In this approach, the precipitation was performed in the same NMR tube where hyperpolarization took place. 400 μL EtAc were added quickly to the sample consisting of 100 μL of 30 mM HP [1-¹³C]pyruvate, right after removing this sample from the 0.4 μT field and releasing the pressure (≈3-4 s). To mix the solution after EtAc addition, further p-H₂ bubbling was used at a low flow rate (˜5-10 scc/m, ambient pressure), FIGS. 5A-5B. For re-dissolution, 300 μL water were added directly to the NMR tube with the precipitate-containing solution, and again, gas flow was used to mix and dissolve the pyruvate microcrystals. After mixing the solutions, most of the aqueous phase rapidly separated from the organic phase (≈1 s). Typical precipitation time was t_{prec}≈2 s and water addition time t_{water}≈6 s, where t_{water} consisted of 1 s to add water, plus 4 s to bubble gas, and 1 s to settle the solution, FIGS. 5A-5B (see FIG. 17 for more information). It was found that adding EtAc in this ratio of 4 to 1 to the HP sample precipitated most of the pyruvate (62±11%, FIG. 17) and after re-dissolution, 39±1% of the HP pyruvate was transferred to the aqueous phase (Table 1). Using this protocol, the obtained two-phase samples visibly contained a major fraction of the catalyst in the organic phase (which remained yellowish and turned reddish after exposure to atmosphere, FIG. 15), whereas a clear aqueous solution of total volume of ≈335±10 μL was obtained (as detailed below approximately half of the CH₃OH remained in the aqueous phase), FIGS. 14A-14B. The total volumes of the investigated mixtures containing the precipitate were approximately 500 μL (3.9 cm fill height in the NMR tube), and after the addition of water the total fluid volume was ≈800 μL (≈6 cm fill height). During the phase mixing, parts of the sample were lifted by approximately 5 cm by the ascending p-H₂ gas bubbles. The sample height in the NMR tube was the decisive factor affecting the choice of solvent volumes to minimize the mixtures from leaving the 1.4 T field of the benchtop NMR spectrometer (approximately 10 cm along the sample tube) as much as possible. At the same time, 300 μL water were added despite significantly diluting the smaller initially polarized 100 μL of 30 mM [1-¹³C]pyruvate batch, FIG. 5A, to fill the sensitive NMR detection volume in the spectrometer to avoid detrimental line broadening that could otherwise affect the quantification. These limitations are clear shortcomings of the current experimental design and can be remedied in the future with purpose-built hyperpolarization equipment.

TABLE 1
NMR analysis of phase-separated solutions

| | 30 mM pyruvate 100 μL MeOH | | | | | | | |
|---|---|---|---|---|---|---|---|---|
| | 400 μL ethyl acetate 300 μL D₂O | | | | | | | |
| | Organic phase | | | | Aqueous phase | | | |
| Sample | EtAc/ | MeOH/ | Pyr/ | Pyr/ | EtAc/ | MeOH/ | Pyr/ | Pyr/ |
| no. | mM | mM | mM^{a} | % | mM | mM | mM^{a} | % |
| 1 | 8532 | 1159 | n.d. | n.d. | 521 | 2011 | 3.2 | 36 |
| 2 | 8804 | 1419 | n.d. | n.d. | 581 | 2445 | 3.7 | 41 |
| 3 | 9062 | 1470 | n.d. | n.d. | 647 | 2838 | 4.1 | 46 |
| 4 | 9102 | 1232 | n.d. | n.d. | 587 | 2294 | 3.2 | 36 |
| 5 | 8856 | 1255 | n.d. | n.d. | 638 | 2823 | 3.7 | 42 |
| 6 | 9284 | 1237 | n.d. | n.d. | 528 | 1960 | 2.7 | 30 |
| 7 | 9263 | 1419 | n.d. | n.d. | 609 | 2472 | 3.7 | 42 |
| 8 | 9288 | 1529 | n.d. | n.d. | 613 | 2479 | 3.3 | 37 |
| 9 | 9302 | 1269 | n.d. | n.d. | 604 | 2527 | 3.0 | 33 |
| 10 | 9174 | 1387 | n.d. | n.d. | 644 | 2688 | 3.4 | 38 |
| Mean ± | 9042 ± | 1316 ± | <0.3 | <5% | 595 ± | 2451 ± | 3.5 ± | 39 ± |
| s.d. | 247 | 102 | mM | | 44 | 300 | 0.4 | 5 |

^{a}Note that initial pyruvate sample was diluted by factor 3.35 (D₂O fraction) to fill the sensitive volume of the benchtop NMR. Likewise, the organic phase (supernatant fraction) had a 4.65-times larger volume compared to the original 100 μL methanol sample. Additionally, for the NMR analysis all samples were diluted by a factor 3 with deuterated solvents. The average pyruvate content in aqueous phase samples was therefore (3.5 ± 0.4), corresponding to 39 ± 5% of the original pyruvate. In other words, if the same amount of pyruvate had been extracted into 100 μL H₂O (without dilution), concentration had been 11.7 ± 1.5 mM. This extraction protocol was later improved as described elsewhere herein.

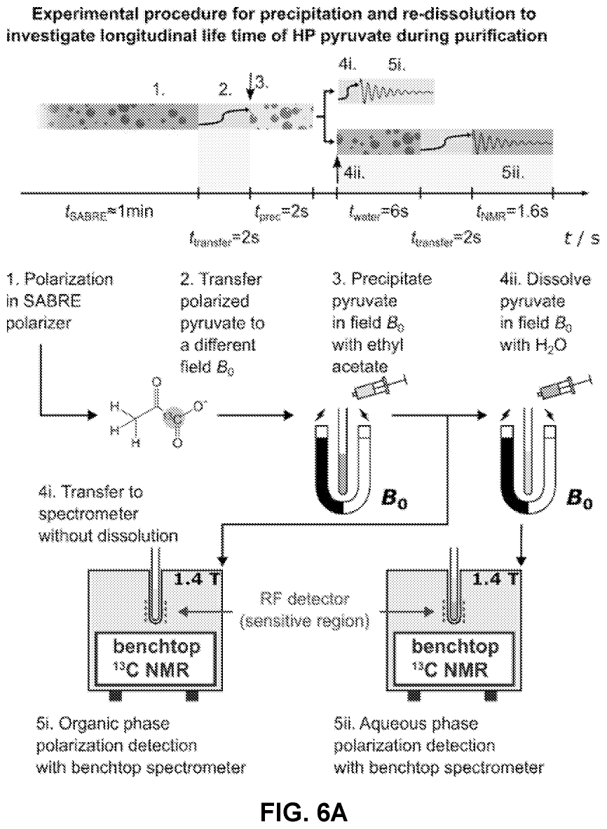

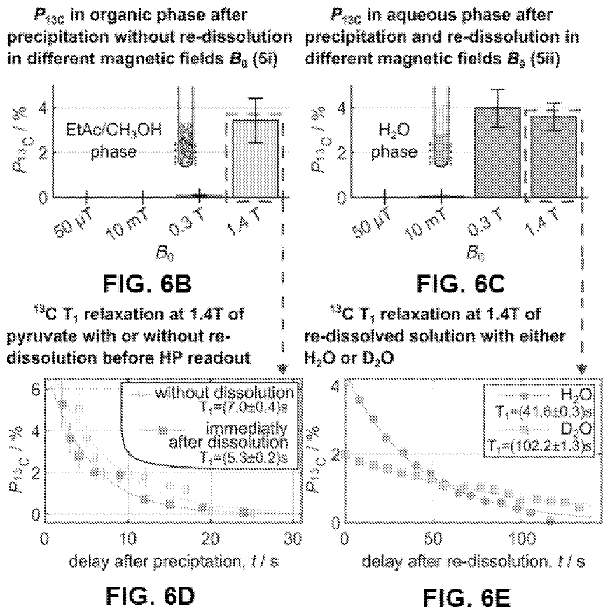

This separation approach was investigated at 4 different magnetic fields: (i) Earth's field; (ii) 10 mT; (iii) 0.3 T; and (iv) 1.4 T, FIGS. 6A-6B. More details on the experiment setups are reported in the SI. Re-D SABRE was performed at (i)-(iv), and the samples were then rapidly transferred to the benchtop 1.4 T NMR, such that the aqueous phase was approximately centered in the radiofrequency coil. In case (iv), for which no transfer was needed, a ≈5-s delay was added to the end of the protocol for consistency with the time needed to transfer the samples in (i-iii).

The detection and quantification of hyperpolarization took place at 1.4 T in all cases. It was expected that low field substantially reduces the hyperpolarization lifetimes in the precipitate, hence, precautions were taken to prevent samples from leaving the studied magnetic fields (i-iv).

No HP ¹³C signal was observed for the samples with dissolution at Earth's field (i), whereas the 10 mT magnet (ii) still provided P_{13C}=0.05±0.02%. In sharp contrast, performing re-dissolution SABRE at the higher fields, (iii) and (iv), provided much higher P_{13C}=4.0±0.8% (N=2) and P_{13C}=3.6±0.6% (N=3), respectively, FIG. 6C (3.5±0.4 mM HP [1-¹³C]pyruvate in the ≈335 μL aqueous sample, i.e., 39±1% of the starting quantity, Table 1). It was concluded that precipitated sample exposure to fields below 0.3 T is detrimental to P_{13C}—in line with the observation of no detectable P_{13C} using a precipitate filtering approach performed at 10 mT fields.

The aqueous solution of HP [1-¹³C]pyruvate contained residual CH₃OH (2.4±0.3 M, i.e., reduced by an order of magnitude ≈2-fold as half the CH₃OH remained in the supernatant and additional 3.35-fold due to dilution) and EtAc (0.8±0.2 M), N=10 samples, Table 1. Elemental analysis revealed 8.5±0.5 ppm mass fraction of Ir metal present in the aqueous phase (Table 2), corresponding to a reduction of Ir concentration by ≈135 fold (by 40-fold due to catalyst partitioning and additional 3.35-fold due to dilution), FIG. 15.

TABLE 2
Iridium Elemental Analysis^{a,b}

| Sample Name | Ir (ppb) | Ir (mM) |
|---|---|---|
| #32 D₂O | 2275 | 0.01 |
| #33 D₂O | 3234 | 0.02 |
| #34 D₂O | 3031 | 0.02 |
| #32 supernatant | 19019 | 0.10 |
| #33 supernatant | 24843 | 0.13 |
| #34 supernatant | 23748 | 0.12 |

| | |
|---|---|
| Detection limit (ppb) | 0.05 |

^{a}Note that initial pyruvate sample was diluted by factor 3.35 (D₂O fraction) to fill the sensitive volume of the benchtop NMR. Likewise, the organic phase (supernatant fraction) had a 4.65-times larger volume compared to the original 100 μL methanol sample (consisting of 30 mM pyruvate and 6 mM Iridium-containing pre-catalyst). Additionally, for the NMR analysis all samples were diluted by a factor 3 with deuterated solvents. The presented numbers refer to the concentrations that were measured in the final diluted solutions
^{b}For example, 2275 ppb value means that the actual Ir content in the non-diluted aqueous sample was 6825.

### Example 6: Lifetime of Polarization in the Precipitated and Aqueous Samples

To investigate the impact of precipitate relaxation in variable magnetic fields during the dissolution process, the Re-D SABRE experiment was repeated, omitting the addition of water after precipitation (i.e., the decay of ¹³C HP signal in the dissolved phase of the precipitated samples was measured). In both settings with low field (i and ii) no measurable ¹³C polarization was observed, FIG. 6B. For (iii), P_{13C} was still much lower compared to the sample that rested at the 1.4 T magnet (setup iv), i.e., P_{13C}=0.08±0.02% and P_{13C}=3.4±0.9%, respectively—this signal is entirely derived from the remaining dissolved pyruvate in solution (2.3±0.6 mM in the 500 μL sample of EtAc and CH₃OH, FIG. 17) not from the precipitated micro-crystalline phase. P_{13C} comparison in setup (iii) and (iv) in FIG. 6B suggests that the dissolved-phase HP [1-¹³C]pyruvate in the EtAc fraction is extremely sensitive to low field exposure: P_{13C} in (iii) was ≈40-times lower than P_{13C} in (iv). However, in FIG. 6C, it was observed that P_{13C} losses are similar when the entire process is contained at 0.3 T (iii) and 1.4 T (iv) revealing that HP [1-¹³C]pyruvate in the aqueous phase is relatively immune to low-field exposure, and microcrystal exposure to fields as low as 0.3 T is not detrimental to P_{13C}. This observation is important because it means the Re-D SABRE process can be successfully performed at storage fields as low as 0.3 T. The rapid ¹³C T₁ of HP [1-¹³C]pyruvate in the EtAc fraction at Earth's field, estimated to be <1.3 s, may be caused by the high catalyst: substrate ratio in the EtAc.

Finding the surprisingly short T₁ in Earth's field, the T₁ of the precipitated sample phases was measured at 1.4 T in configuration (iv). HP signals from the sample before or after re-dissolution were measured as a function of an additional delay t_{d} (ranging from 0 to 23 s; t=t_{prec}+t_{d}), during which P_{13C} was allowed to decay. At the end of t_{d}, the signal was detected either immediately (green circles, FIG. 6D, sampling the decay rate of the residual dissolved-phase pyruvate) or after the dissolution with D₂O (squares, FIG. 6D, sampling the decay rate of the entire pyruvate (precipitated and dissolved) pool). Assuming a mono-exponential decay, the apparent ¹³C T₁ was 7.0±0.4 s or 5.3±0.2 s, respectively. These T₁ times are surprisingly short and deserve future detailed investigations. In sharp contrast, T₁ values in the aqueous phase measured after the Re-D SABRE process were 41.6±0.3 s in H₂O and 102±1.3 s in D₂O, FIG. 6E, using a series of pulse-detect NMR experiments with low flip angle of 9° (N=2).

Naturally, a longer T₁ in the precipitated phase would be helpful to retain a higher fraction of produced hyperpolarization. Hence, future investigations should consider evaluation of T₁ at different temperatures, magnetic fields, residual O₂ content, precipitating solvent composition, or as function of pH. Although high Re-D SABRE polarizations were obtained above (FIGS. 6A-6B), on average only ≈39% of the initial [1-¹³C]pyruvate was transferred into the aqueous phase in that protocol (determined by high-resolution NMR, Table 1). This observation may be attributable to an inefficient mixing of phases.

### Example 7: Maximizing P_{13C} and Extraction of Aqueous [1-¹³C]Pyruvate and Trace Analysis of the Obtained Solutions

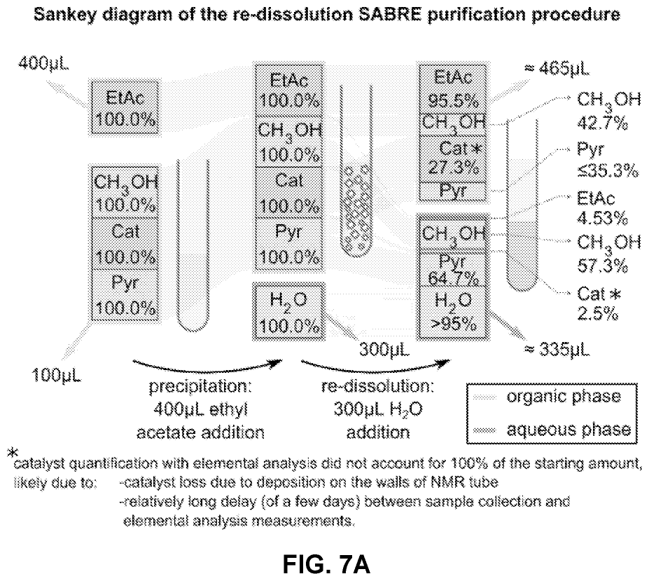

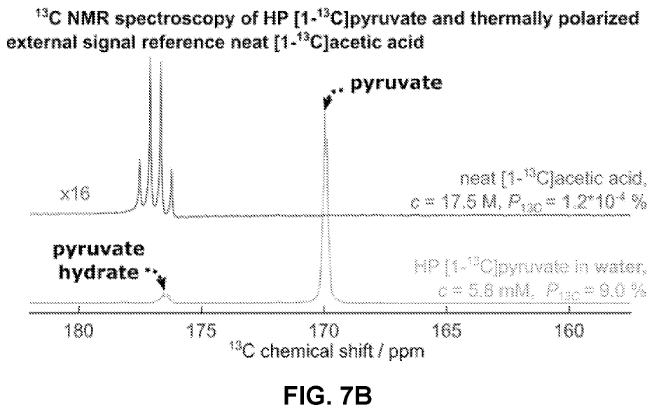

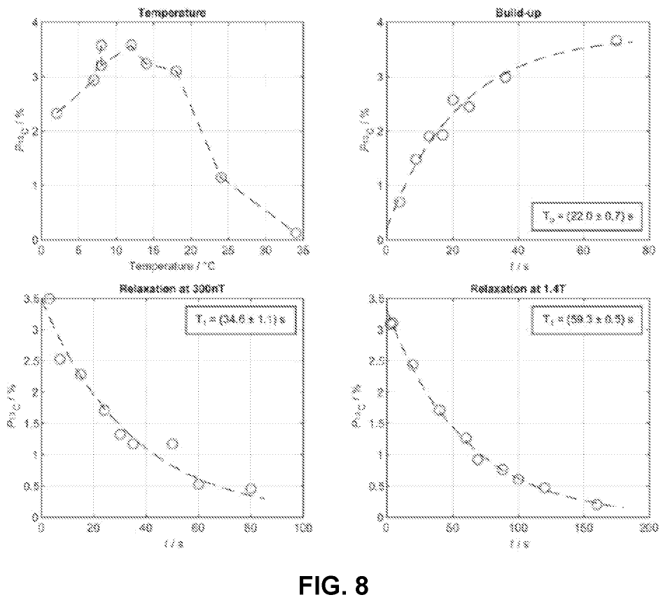

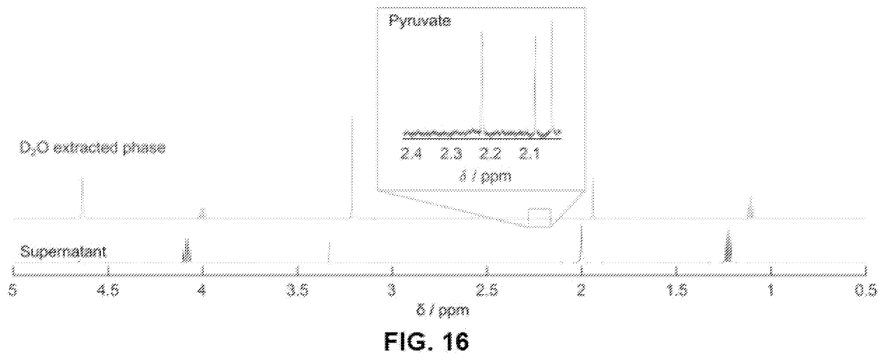

A more rigorous phase mixing protocol was tested, where the bubbling tube was moved up and down rapidly three times for phase mixing during the t_{water}≈6 s mixing time. Using this procedure (N=2), 65±7% of the initial pyruvate were extracted (vs. ≈39% as described above) into the aqueous phase, as confirmed by high-resolution NMR spectroscopy, FIG. 16. Considering the final volume of the aqueous phase of 335±10 μL and the measured pyruvate concentration of 5.8±0.8 mM (in the diluted Re-D SABRE sample), the detected ¹³C signals corresponded to a nuclear polarization of P_{13C}=9±1% or 8±1%, i.e., ε_{13C} of ≈75,000 at 1.4 T, respectively, N=2 samples, FIGS. 7A-8B.

The reproducibility of the described SABRE-SHEATH polarization experiment should be noted, which exhibits large statistical deviations (maximum of P_{13C}=14.5%, but 8±2% was the inter-day mean with standard deviation). Future setup automation of the hyperpolarization and sample shuttling procedure and more standardized experimental and sample preparation protocols are anticipated to improve the reproducibility and mean P_{13C} values before and after HP [1-¹³C]pyruvate sample purification.

Based on the presented pilot studies, several possible improvements are envisioned to yield more concentrated and polarized HP [1-¹³C]pyruvate with lower concentration of the residual solvent for the future Re-D SABRE implementations.

First, SABRE-SHEATH hyperpolarization can also be effective at higher [1-¹³C]pyruvate concentration in the initial CH₃OH solution, e.g., at 60 mM as demonstrated recently.

Second, a larger hosting magnet can enable the use of a larger purpose-built reactor to potentially allow improving the ratio of initial volume of polarized [1-¹³C]pyruvate solution to the final aqueous HP sample volume to yield higher HP [1-¹³C]pyruvate concentration in the final aqueous solution. Also, a larger reactor may allow adding more EtAc to achieve a greater EtAc: CH₃OH ratio for more efficient pyruvate precipitation from the supernatant, and also to absorb more CH₃OH to reduce its concentration in the final aqueous solution, see Table 3 below.

TABLE 3
Substance concentrations found by high-resolution NMR analysis of syringe-filtered solutions^{a}

| | | 30 mM pyruvate in 500 μL MeOH crushed in | | | | | | | | | |
|---|---|---|---|---|---|---|---|---|---|---|---|
| EtOH | | Acetone | | | | | Ethyl Acetate (EtAc) | | | | |
| washing | Volume/ | Acetone/ | Methanol/ | Ethanol/ | Pyruvate | | EtAc/ | Methanol/ | Ethanol/ | Pyruvate | |
| (5 mL) | mL | mM | mM | mM | mM | % | mM | mM | mM | mM | % |
| No | 2 | 2683 | 623 | — | 0.9 | 3.0 | 556 | 689 | — | 6.5 | 21.5 |
| | 4.5 | 846^{b} | 95^{b} | — | 0.2 | 0.7 | 449 | 262 | — | 4.4 | 14.6 |
| | 9.5 | 3458 | 184 | — | 4.4 | 14.8 | 459 | 166 | — | 8.9 | 29.7 |
| Yes | 2 | 75 | 22 | 959 | 0.1 | 0.4 | 28 | 22 | 3638 | 8.6 | 28.5 |
| | 4.5 | 30 | 5 | 2575 | 6.8 | 22.5 | 16 | 6 | 3610 | 6.0 | 20.0 |
| | 9.5 | 123^{b} | 7^{b} | 4795^{b} | 3.3 | 10.9 | 17 | 3 | 4397 | 12.7 | 42.2 |

| | | | | | | | | |
|---|---|---|---|---|---|---|---|---|
| No | 4.5 | Precipitating in 4.5 mL EtAc was | D₂O | 302 | 336 | — | 6.0 | |
| | | re-peated without EtOH washing and | supernatant | 7917 | 2157 | — | 0.4-2^{c} | 1-5^{c} |

supernatant solution was analyzed

^{a}Note, for stated pyruvate concentrations, the CH₃OH—H2O ratio (1:2) was considered for comprehensibility (i.e., c_{PYR} in the measured samples were 2-fold lower
^{b}Filter broken
^{c}In this spectrum, signal-to-noise ratio was too poor to be more precise

Third, rapid filtering of the precipitate appears to be a promising alternative to the phase separation method employed here. Indeed, initial tests were performed with commercial 0.2 μm syringe filters and cotton-filled syringe-based filtration columns clearly showing that most (>99%) of the organic solvent and catalyst can be effectively removed and the precipitate pyruvate can be recovered in neat water after re-dissolution from the filter. However, the manual implementation of these two filtration alternatives consumed 1-2 minutes when performed manually, and therefore, HP ¹³C signals were not observed with these approaches, consistent with the results presented herein.

Fourth, the relatively short ¹³C T₁ in the precipitate implies that future efficient precipitation and re-dissolution procedures would need to be performed quickly on the time scale of a few seconds. Thus, rapid automated purification of the samples can potentially minimize T₁-associated polarization losses, and therefore, yield a better polarized product with less contaminations. Note that rapid purifications have been already established in the literature for hydrogenative PHIP techniques and allowed production of solutions, in which traces of solvents, catalyst, and side products of the hydrogenation were below recommended limits for patient applications.^{[24,44,45]} Hence, these other promising advances bode well for producing biocompatible HP [1-¹³C]pyruvate via the presented Re-D SABRE approach for widespread, cost- and time-efficient preclinical and clinical future applications.

Conclusion: It has been demonstrated that Re-Dissolution (Re-D, pronounced “ready”) SABRE-SHEATH efficiently enables extracting HP [1-¹³C]pyruvate into an aqueous phase with P_{13C} of up to 9%. This approach shines in its simplicity since no hydrogenation reaction or any chemical modification of the substrate molecule is needed for this technique compared to PHIP-SAH. Moreover, the presented approach is substantially faster compared to the leading d-DNP technique. While the approach would likely benefit from further refinement of the protocol (e.g., further reduction of solvent/catalyst content and improvement of degree of polarization) for in vivo applications, it is anticipated that future automation efforts (using already demonstrated approaches for different PHIP techniques^{[44]}) will provide a robust procedure to prepare biocompatible HP [1-¹³C]pyruvate formulations for use in next-generation molecular imaging modalities that are both affordable and accessible for clinical utilization.

### Example 8: Further Experimental Considerations

Preparation of samples: Parahydrogen was enriched to >99.10% and filled into aluminum cylinders at 350 psi using a setup described previously. The SABRE samples were prepared in CD₃OD (CAS: DLM-24-25), CH₃OH (CAS: 67-56-1), or ethanol (CAS: 64-17-5) using the stated concentration of sodium pyruvate (in most cases 30 mM, natural abundance—CAS: 113-24-6; [1-¹³C]-labeled—CAS: 490709-250 MG), an iridium catalyst at 6 mM concentration synthesized as described previously, doped with 40 mM dimethyl sulfoxide (DMSO) to block one active center of the catalyst and to avoid pyruvate from binding to the catalyst at two sites. Oxygen was removed from the samples by guiding argon gas at approximately ambient pressure through the samples for ˜1 min. Also, oxygen was removed from all solvents used for the preparation of the samples (methanol or ethanol) or for the precipitation of pyruvate (ethyl acetate (EtAc), room temperature, CAS: 141-78-6) prior to mixing them to the catalyst substrate solutions. For the SABRE experiments, the samples were filled into medium wall, or in case of precipitation studies, into regular wall NMR tubes (SKU: WG-1000-8, outer diameter 5 mm, inner diameter 4 mm and length 8 inches). Note that the total volume of the catalyst-substrate sample was 600 μL (in the baseline and solvent study) or 100 μL in experiments were precipitation and phase separation were performed. In case of dissolution SABRE, 300 μL of room-temperature H₂O (HPLC grade-submicron filtered) or D₂O (CAS: 7789-20-0) were added.

SABRE hyperpolarization setup: The experimental setup has been used previously but will be described in much greater detail elsewhere. Briefly, it consisted of a 3-layer mu-metal of 3″ I.D. and 9″ DEPTH to shield external magnetic fields (Magnetic Shield Corporation, ZG-203), combined with a custom-made solenoid to generate a static magnetic field, B0, of ˜300 nT (28 AWG wire, 650 windings, 7″ homogeneous length). Note that this coil provided a constant magnetic field here (5 VDC power supply connected to the solenoid via a resistor board to regulate the current and effectively set the magnetic field).

The setup for bubbling parahydrogen through the sample tubes consisted of a mass flow controller, which regulated the flow of pressurized parahydrogen, a safety valve which released pressure above 100 psi from the setup, and a combination of manual valves as depicted in Scheme 3. Parahydrogen was guided through the samples from the bottom of the NMR tube using a tube (OD×ID of 1/16″× 1/32″), when the bypass valve was closed. An additional valve allowed for rapid venting of the overpressure from the system and NMR tubes, e.g., for opening the tubes and filling additional solvents for precipitation of dissolution SABRE experiments. Note that in contrast to the previous reports; here the mass flow controller was bypassed to be able to provide a low, constant flow of parahydrogen to mix solutions in the precipitation and dissolution SABRE studies, as described elsewhere herein. To regulate the flow, a high-precision threaded flow-adjustment valve (McMaster Carr, P/N 7832K22) was mounted in the flow path parallel to the mass flow controller (SmartTrak 50, Sierra Instruments).

¹³C polarization detection and quantification: Hyperpolarized and thermal NMR signals were measured using a 1.4 T benchtop NMR system (Spinsolve 60 Carbon, Magritek, Germany). Hyperpolarization PHP was quantified by comparing the hyperpolarized signal with a thermally-polarized reference (with polarization Pref) of ¹³C-labeled acetic acid and accounting for differences in concentration, c, and NMR tube inner cross-section, A (if different NMR tubes were used), using the following formula:

$${P}_{HP} = {P}_{ref} \cdot \frac{{S}_{HP}}{{S}_{ref}} \cdot \frac{{F}_{13 C , ref}}{{F}_{13 C , HP}} \cdot \frac{{c}_{ref}}{{c}_{HP}} \cdot \frac{{A}_{ref}}{{A}_{HP}}$$

HP and ref refer to the hyperpolarized and reference sample, respectively, S is the measured signal, and F_{13C} is the fraction of ¹³C labeling.

Measurement of solution compositions using high-resolution ¹H NMR spectroscopy: Once the samples were prepared, thermal ¹H NMR signals of the solvent (e.g., CH₃OH, EtAc, etc.) were measured using a 1.4 T benchtop NMR system (Spinsolve 60 Carbon, Magritek, Germany), and quantified using external signal reference (thermally polarized neat [1-¹³C]acetic acid). This approach allowed the performance of quantification of residual solvent in millimolar concentration. Next, the samples were taken to high-resolution NMR spectrometer (500 MHz or 600 MHz) for 1H NMR spectra acquisition to perform detection of solvent peaks as well as residual pyruvate (see examples of the spectroscopy herein). Using the concentrations of the residual solvent, the pyruvate concentration was determined. In selected number of samples, ¹³C NMR spectroscopy (and external ¹³C signal reference sample) has also been employed to independently confirm that the measured concentration (by ¹H NMR) matched that determined by ¹³C NMR spectroscopy to avoid systematic errors. Long recovery times were employed (>1 minute) to ensure the samples gained thermal equilibrium polarization.

For SABRE experiments, the sample NMR tubes were mounted to the bubbling setup and pressurized to 100 psi. Subsequently, a parahydrogen flow of 75 scc/m was provided. Activation of the catalyst took place for 3 min at ambient temperature and magnetic field. For polarization build up, if not stated otherwise, the sample was placed in the 0.30 μT field and a water bath of the desired temperature (typically between 10-15° C.).

Data fitting and error propagation for relaxation and polarization build-up data presented in FIGS. 4A-4I: Data in FIGS. 4C, 4F, 4H was fitted using mono-exponential functions (solid lines) and extracted values with error of polarization build-up or relaxation are presented in bar plots (FIGS. 4D, 4E, 4G, 4I). Mean and standard error of the maximum/averaged polarization obtained in the investigated solvents are presented in FIG. 4D.

Error propagation for precipitation and dissolution SABRE at different magnetic fields shown in FIGS. 6A-6E: Presented error bars in FIGS. 7A-7B were propagated from estimated errors of sample volumes (±10 μL), and measured values with error for the concentrations in the respective phases. In FIG. 6D, data at 0.3 T and 1.4 T was measured N=2 or 3 times and mean values with errors propagated from the errors of the single experiments are presented.

SABRE-SHEATH hyperpolarization of [1-¹³C]pyruvate at 60 mM concentration: Production of samples with even higher concentration of [1-¹³C]pyruvate in methanol was explored but it was found that with current instrumentation and SABRE-SHEATH protocol, this approach is unfavorable. Specifically, using 60 mM [1-¹³C]pyruvate and the same catalyst concentration and p-H₂ flow rate (6 mM and 80 scc/m, respectively) in CH₃OH, P_{13C} was 3.7±0.06%, FIG. 8.

Description of the magnets used in the precipitation and relaxation study: The precipitation and phase separation approach was investigated at 4 different magnetic fields: (i) Earth's field; (ii) 10 mT; (iii) 0.3 T; and (iv) 1.4 T. For (i), the samples were positioned in a plastic holder at a defined position in the laboratory at which a magnetic field of ≈40 μT was measured. For (ii), a modified setup of permanent magnets creating a 10 mT magnetic field over a volume of ≈20×10×10 cm³ was employed. For (iii), a hollow cylindrical magnet (K&J Magnetics, Inc., P/N RY04Y0) was used with a length of 5.1 cm and an inner bore of approximately 6.4 mm diameter in which the NMR tube was positioned. For (iv), the NMR benchtop spectrometer was used, which approximately provides the constant 1.4 T field up to 10 cm above the magnet center.

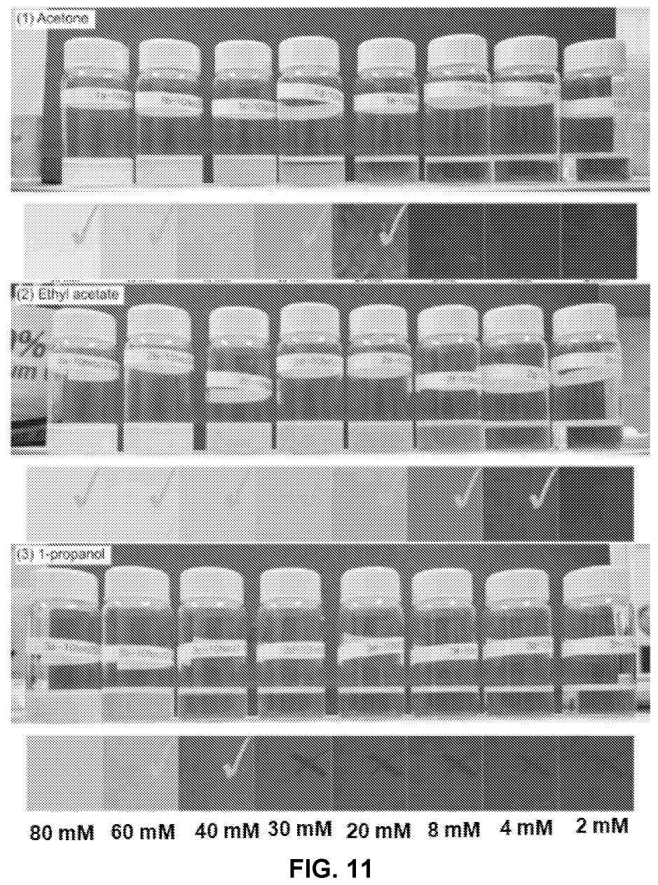

Precipitating and filtering of pyruvate from solutions using thermally polarized natural-abundance sodium pyruvate: Considering the finding of low pyruvate solubility herein, ethanol was an obvious first choice as the precipitating solvent. However, it was found that even when a 9-fold volume of ethanol was added to 80-mM sodium pyruvate in methanol solutions (i.e., a dilution factor of ≈10), no precipitation was detected. Other candidates (acetone, 1-propanol, and ethyl acetate) were tested next. All three solvents mixed with methanol and dissolved 6 mM of SABRE catalyst well. Hence, a titration study of the solubility of natural-abundance sodium pyruvate was performed (CAS: 113-24-6). For each of the three solvents (FIG. 11) eight 500-μL batches of methanol containing different concentrations of pyruvate were prepared (c_{pyr}∈[80, 60, 40, 30, 20, 8, 4, 2] mM). Then, 9.5 mL of the respective precipitating solvent were added to each sample. In all 80 mM samples, prominent and almost instantaneous precipitation was observed as the solutions lost transparency and became milky. For 1-propanol, acetone, and ethyl acetate, precipitation was visible for all mixtures with 40 mM, 20 mM, and 4 mM or more initial concentration of pyruvate, respectively. Additionally, as ethyl acetate and water are immiscible, a later purification of this solvent was expected to be advantageous (see below). Based on these observations, the rest of the study was continued with acetone and ethyl acetate.

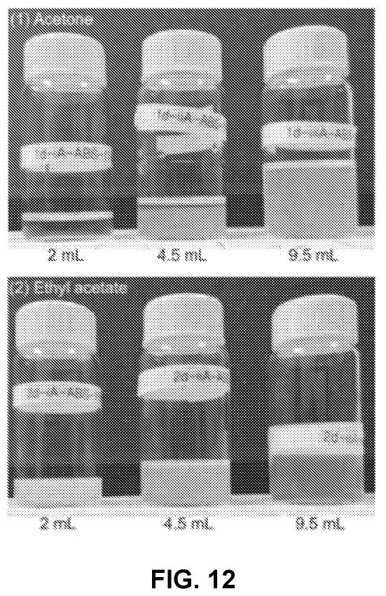

For acetone and ethyl acetate, the dilution factor used was varied, i.e., different amounts of the precipitating solvent (2 mL, 4.5 mL, or 9.5 mL) were added to 500 μL samples of 30 mM pyruvate in methanol. In all samples, precipitating of pyruvate was observed, but in line with the previous observation, the pyruvate precipitation was more apparent in the ethyl acetate samples (FIG. 12).

Excited by these findings, and as a promising alternative to the phase separation approach described elsewhere herein, filtering of non-hyperpolarized pyruvate from the precipitated solutions was tested and the obtained solutions were analyzed with high resolution NMR. To this end, syringes were filled with 500 μL CH₃OH with 30 mM unlabeled pyruvate and the respective precipitating solvent was loaded into the syringe. Again, variable precipitating solvent volume was added to each sample (2, 4.5, or 9.5 mL acetone or ethyl acetate), i.e., to dilute the initial pyruvate-containing solutions by a factor of 5, 10, or 20. Subsequently, a commercially-available syringe filter (pore size 0.22 μm, sterile hydrophilic Fisher-brand filter, P/N 09719C) was mounted to the tip of the syringe and the solution was slowly manually ejected into a vial at a flow rate of approximately 0.5 mL/s. This procedure was followed by flushing 10 mL air from a syringe through the filter to remove more residual solvent. Last, 1 mL D₂O was guided through the filter at the same flow rate to dissolve the solidified pyruvate which was caught by the filter. The same experiment was repeated, but before passing the D₂O, 5 mL ethanol were flushed through the filter to wash out the less biocompatible solvents and catalyst additionally.

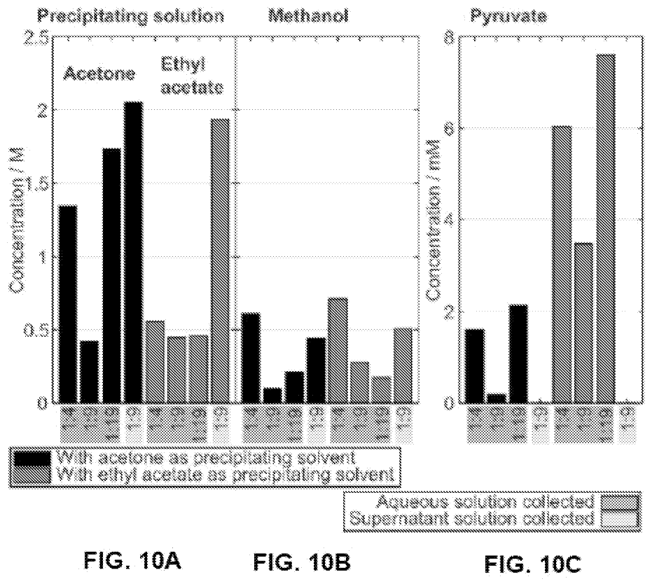

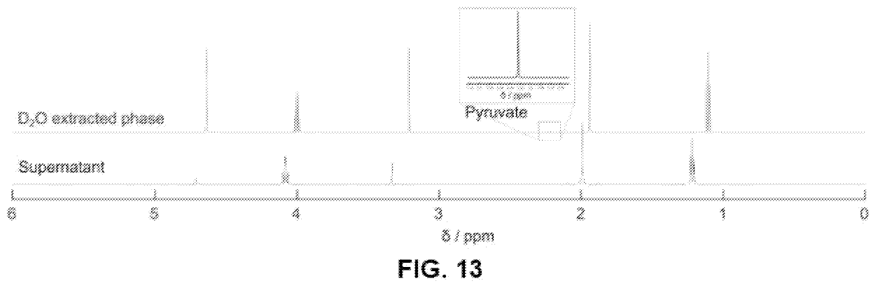

NMR analysis of the supernatant (acetone/ethyl acetate-catalyst-methanol mixtures), flushed ethanol, and D₂O solutions showed that pyruvate can be detected quantitatively in the present samples: although no clear trend of concentrations of solvents and pyruvate with the dilution factor was observed and concentration standard deviations were relatively large, ethyl acetate performed clearly better than acetone: in the D₂O samples obtained after acetone precipitating, (6±4) % of the original pyruvate was detected compared to (22±4) % for ethyl acetate (1.9±1.3 mM vs. 6.6±1.9 mM after accounting for the different volumes of initial methanol, 600 μL, and D₂O, 1 mL). Moreover, the traces of the precipitating solvents in D₂O samples were significantly higher in the acetone precipitated solutions (2329±774 mM) compared to ethyl acetate (488±34 mM), while the residual concentration of methanol was similar (301±163 mM vs. 372±161 mM respectively). The residual solvent concentrations were most likely caused by the large inner volume of the syringe filters (≈500 μL). Interestingly, remaining pyruvate concentrations in the supernatant solutions was low and estimated to 1-5% of the original pyruvate (estimated error is large because of poor signal-to-noise ratio in the spectra of the supernatant; in these samples the initial 0.6 mL sample size was first diluted by a factor of 5-20 by the precipitation, and then another factor of 5 by deuterated solvent for high-resolution NMR; Table 3, FIGS. 10A-10C, FIG. 13).

Regarding the additional washing of precipitated sodium pyruvate with ethanol, this method performed very well in terms of removing the supernatant solutions from the filter (e.g., in the ethyl acetate precipitated solutions, methanol content was further reduced to (10±6) mM and ethyl acetate to (20±5) mM). At the same time, the mean pyruvate fraction obtained in D₂O after the ethanol washing was slightly higher than in samples without ethanol washing (30±6% in ethyl acetate samples; 11±6% in acetone samples).

As an alternative to the syringe filters, filtering ethyl-acetate-precipitated solutions through a column filled with cotton was also tested, and led to similar results (Table 4). Note that both cotton and syringe filtering, were performed manually without any automation of the procedure.

TABLE 4
NMR Analysis of Cotton-Filtered Solutions^{a}

| 30 mM pyruvate in 600 μL | | | | | |
|---|---|---|---|---|---|
| MeOH crushed in | | Ethyl Acetate (EtAc) | | | |
| Volume/ | | | Methanol/ | Pyruvate | |
| mL | sample | EtAc/mM | mM | mM | % |
| 2 | Supernat. | 6332 | 5145 | 2.2 | 12 |
| | 1^{st} D₂O | 264 | 258 | 2.4 | 13 |
| | 2^{nd} D₂O | 17 | 12 | 0.2 | 1 |
| | 3^{rd} D₂O | n.d. | n.d. | n.d. | — |
| 4.5 | Supernat. | 7029 | 2277 | <0.1 | 0 |
| | 1^{st} D₂O | 624 | 536 | 5.7 | 32 |
| | 2^{nd} D₂O | 181 | 45 | 1.1 | 6 |
| | 3^{rd} D₂O | 19 | n.d. | 0.1 | 1 |
| 9.5 | Supernat. | 9004 | 1416 | <0.1 | 0 |
| | 1^{st} D₂O | 564 | 420 | 6.6 | 37 |
| | 2^{nd} D₂O | 248 | 74 | 1.9 | 10 |
| | 3^{rd} D₂O | 45 | 15 | 0.7 | 4 |

^{a}Note that after filtering the supernatant solution through the cotton filter, three samples of 1 mL D₂O were filled and passed through the cotton subsequently to investigate if dissolution of pyruvate salt was incomplete. Pyruvate in [%] refers to fraction of initial 30 mM pyruvate in 600 μL CH₃OH and accounts for dilution in larger D₂O samples (i.e., 1 mL D₂O/0.6 mL CH₃OH.

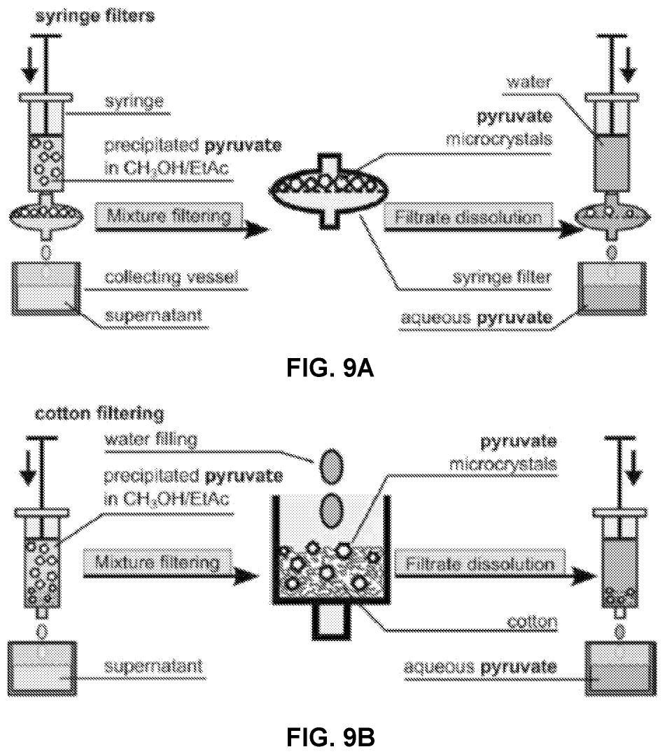

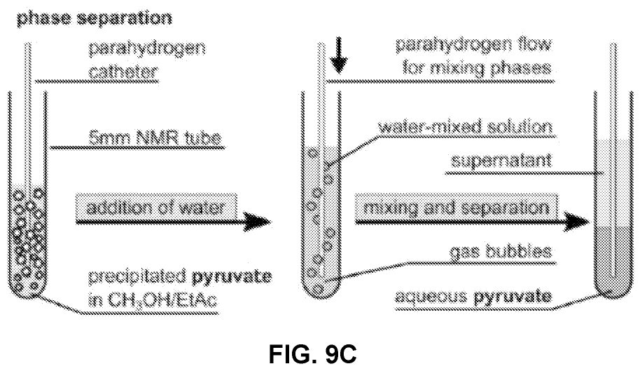

As described above, filtering of non-hyperpolarized pyruvate from the precipitated solutions was tested using in-line syringe filters (FIGS. 9A-9C), and the obtained solutions of supernatant and dissolved microcrystals were analyzed with high resolution NMR. Cotton filters were also used as a performance metric for the syringe filters. Each sample was SABRE-SHEATH-polarized as described previously for three times to quantify the mean base-line hyperpolarization before precipitation (for this batch of samples, P_{13C}=10±1% in CH₃OH). Because a short lifetime of ¹³C magnetization was expected at ultralow field, the precipitation procedure was performed at an elevated field of 10 mT (see setup described in section 5). At this field, proton and ¹³C Zeeman levels are separated by >350 kHz, i.e., an order of magnitude greater than the dipolar spin-spin couplings present in the solid state. To this end, after the hyperpolarization procedure was completed, the sample was rapidly removed from the 0.30 μT field, depressurized, and filled into a 10-mL plastic syringe held at a 10 mT field mounted to a Luer-locked filter. After depressurization (˜10-15 s after hyperpolarization) 4.5 mL EtAc was added, the solution was stirred manually, and the filtration was started, taking a total of about 1 min following hyperpolarization. Subsequently, 1 mL of H₂O was guided through the filter, ejecting the solution into a 5-mm NMR tube, which took another ˜20 s regardless of the procedure. Then, the sample was transferred to the adjacent 1.4 T benchtop NMR spectrometer (3-5 s). The whole precipitation and filtering procedure took approximately 90-100 s. The procedure was repeated three times, but no HP ¹³C signal was observed in any sample, requiring the procedure to be altered to apply a more minimalistic approach to signal measurement (see below). This observation is in line with the relaxation study (FIG. 6B), which indicates that [1-¹³C]-T₁ relaxation time of pyruvate at 10 mT in the precipitated solutions was too short—substantially shorter than that of dissolved pyruvate (e.g., 1-¹³C T₁=46.5±0.9 s in CH₃OH: catalyst mixture at Earth's field).

Phase separation approach: As described herein, phase separation was used to extract HP [1-¹³C]pyruvate from methanol to aqueous media. FIGS. 14A-14B show the photos of the samples before and after phase extraction. Because the catalyst changes color upon exposure to air, show in a photo of the phase-extraction samples taken several hours after the procedure (FIG. 15); see the excellent visual coloring of the organic layer at the top indicating that most of the catalyst is indeed retained in the organic layer. An example NMR analysis spectrum used for substance concentration quantification is shown in FIG. 16 with the summary of the results presented in Table 1. The iridium content quantification was performed using elemental analysis, and the data is summarized for organic (supernatant) and aqueous phases in Table 2. The Sankey diagram summarizing the results of the phase extraction studies is presented in FIGS. 7A-7B. The optimization of timings of the sequence for phase extraction is shown in FIG. 17.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Adams, R. W., Science 2009, 323, 1708-1711.
2. Adelabu, I., ChemPhysChem 2022, 23, e202100839.
3. Albers, M. J., Cancer Res. 2008, 68, 8607-8615.
4. Ardenkjær-Larsen, J. H., Magn. Reson. Med. 2019, 81, 2184-2194.
5. Ardenkjær-Larsen, J. H., Proc. Natl. Acad. Sci. 2003, 100, 10158-10163.
6. Barskiy, D. A., J. Am. Chem. Soc. 2014, 136, 3322-3325.
7. Barskiy, D. A., J. Phys. Chem. Lett. 2018, 9, 2721-2724.
8. Bondar, O., Catal. Today 2021, DOI 10.1016/j.cattod.2021.11.030.
9. Bowers, C. R., J. Am. Chem. Soc. 1987, 109, 5541-5542.
10. Carrera, C., ChemPhysChem 2021, 22, 1042-1048.
11. Cavallari, E. Sci. Rep. 2018, 8, 8366.
12. Cavallari, E., Chemphyschem Eur. J. Chem. Phys. Phys. Chem. 2019, 20, 318-325.
13. Chapman, B., Anal. Chem. 2021, 93, 8476-8483.
14. Cheng, T. Comment, NMR Biomed. 2020, 33, e4264.
15. Coffey, A. M., Anal. Chem. 2016, 88, 8279-8288.
16. Colell, J. F. P., J. Am. Chem. Soc. 2017, 139, 7761-7767.
17. Cowley, M. J., J. Am. Chem. Soc. 2011, 133, 6134-6137.
18. Dagys, L., Analyst 2021, 146, 1772-1778.
19. Day, S. E., Nat. Med. 2007, 13, 1382-1387.
20. Ding, Y., Chemistry-Methods n.d., n/a, e202200023.
21. Eills, J., ChemPhysChem 2018, 19, 40-44.
22. Eisenschmid, T. C., J. Am. Chem. Soc. 1987, 109, 8089-8091.
23. Golman, K., Proc. Natl. Acad. Sci. 2006, 103, 11270-11275.
24. Hale, W. G., ChemPhysChem 2021, 22, 822-827.
25. Hövener, J.-B., Anal. Chem. 2014, 86, 1767-1774.
26. Iali, W, Angew. Chem. Int. Ed. 2019, 58, 10271-10275.
27. Iali, W., Angew. Chem. 2019, 131, 10377-10381.
28. Jannin, S., Chem. Phys. Lett. 2012, 549, 99-102.
29. Joalland, B, Anal. Chem. 2020, 92, 1340-1345.
30. Joalland, B., Magn. Reson. Chem. 2021, 59, 1180-1186.
31. Kidd, B. E., J. Phys. Chem. C 2018, 122, 16848-16852.
32. Knecht, S., Proc. Natl. Acad. Sci. 2021, 118, DOI 10.1073/pnas.2025383118.
33. Korchak, S., ChemistryOpen 2018, 7, 672-676.
34. Kurhanewicz, J., Neoplasia 2019, 21, 1-16.
35. Lin, K., ChemPhysChem 2021, 22, 1947-1957.
36. Mamone, S., Angew. Chem. n.d., n/a, DOI 10.1002/ange.202206298.
37. Manoharan, A., ChemMedChem 2018, 13, 352-359.
38. Müller, C. A., NMR Biomed. 2020, 33, e4291.
39. Reineri, F. Nat. Commun. 2015, 6, 5858.
40. Schmidt, A. B., Anal. Chem. 2022, 94, 479-502.
41. Schmidt, A. B., Commun. Chem. 2022, 5, 1-7.
42. Schmidt, A. B., Phys. Chem. Chem. Phys. 2021, DOI 10.1039/D1CP04153C.
43. Shchepin, R. V., Anal. Chem. 2014, 86, 5601-5605.
44. Shi, F., J. Phys. Chem. C 2016, 120, 12149-12156.
45. Skinner, J. G., Mol. Imaging Biol. 2018, 20, 902-918.
46. Spannring, P., Chem. —Eur. J. 2016, 22, 9277-9282.
47. Theis, T., J. Am. Chem. Soc. 2015, 137, 1404-1407.
48. Tickner, B. J., Catal. Sci. Tech. 2020, 10, 1343-1355.
49. TomHon, P., J. Am. Chem. Soc. 2022, 144, 282-287.
50. Truong, M. L., J. Phys. Chem. B 2014, 118, 13882-13889.
51. Vazquez-Serrano, L. D., Inorg. Chim. Acta 2006, 359, 2786-2797.
52. Zeng, H., J. Magn. Reson. 2014, 246, 119-121.

1. A method for preparing a precipitated hyperpolarized substrate, the method comprising contacting a solution comprising a first solvent and a hyperpolarized substrate with a non-polar organic solvent.

2. The method of claim 1, wherein the non-polar organic solvent comprises an unpolarized but otherwise identical substrate in a concentration of from about 1 μM to about 100 mM.

3. The method of claim 1, further comprising separating the precipitated hyperpolarized substrate from the first solvent, the non-polar organic solvent, or any combination thereof.

4. The method of claim 3, wherein separating is accomplished by filtration.

5. The method of claim 4, wherein filtration is carried out using a C18 silica filter, a C9 silica filter, a micro-scale filter, a cellulose acetate filter, a cotton filter, or any combination thereof.

6. The method of claim 2, further comprising redissolving the precipitated hyperpolarized substrate in a biocompatible solvent.

7. The method of claim 6, wherein the biocompatible solvent comprises water or a physiologically-acceptable buffer.

8. The method of claim 7, wherein the physiologically-acceptable buffer comprises saline, phosphate buffered saline, sodium or potassium phosphate buffer, bicarbonate buffer, 2-(N-morpholino)ethanesulfonic acid (MES), bis-(2-hydroxyethyl) amino-tris(hydroxymethyl) methane (Bis-Tris), N-(2-acetamido)iminodiacetic acid (ADA), N-(carbamoylmethyl)-2-aminoethane sulfonic acid (ACES), 2-[4-(2-sulfoethyl)piperazin-1-yl]ethanesulfonic acid (PIPES), 3-morpholino-2-hydroxypropanesulfonic acid (MOPSO), N,N-bis(2-hydroxyethyl)taurine (BES), 3-(N-morpholino)propanesulfonic acid (MOPS), 2-[4-(2-hydroxyethyl)piperazin-1-yl]ethanesulfonic acid (HEPES), N-[tris(hydroxymethyl)methyl]2-aminoethanesulfonic acid (TES), 2-amino-2-(hydroxymethyl)-1,3-propanediol (Tris), N-(hydroxyethyl) piperazine-N′-2-hydroxypropanesulfonic acid (HEPPSO), N-[tris(hydroxymethyl)methyl]glycine (Tricine), N,N-bis(hydroxyethyl)glycine (Bicine), N-[tris(hydroxymethyl)methyl]-3-aminopropanesulfonic acid (TAPS), boric acid buffer, N-cyclohexyltaurine (CHES), or any combination thereof.

9. The method of claim 1, wherein the non-polar organic solvent comprises chloroform, diethyl ether, ethyl acetate, acetone, ethanol, acetic acid, dichloromethane, toluene, xylene, a perfluoropolyether solvent, a hydrofluoroether solvent, a methylsiloxane, a C4-C10 alkane or cycloalkane, or any combination thereof.

10. (canceled)

11. The method of claim 1, wherein the precipitated hyperpolarized substrate comprises pyruvate, oxaloglutarate, oxaloacetate, phenyl pyruvate, 2-oxo-butyrate, 2-oxoglutarate, urea, 2,3-diketogluatarate, 2-oxo-adipate, acetonitrile, benzonitrile, α-cyano-4-hydroxycinnamic acid (CHCA), alectinib, metronidazole, dichloropyridazine, nicotinamide, imidazole, adenine, diphenyldiazene, diazirine, or any combination thereof.

12. The method of claim 1, wherein the precipitated hyperpolarized substrate is hyperpolarized on at least one nucleus selected from ¹H, ¹⁵N, ¹³C, or any combination thereof.

13. The method of claim 1, wherein prior to performing the method, the hyperpolarized substrate is produced using signal amplification by reversible exchange (SABRE).

14.-25. (canceled)

26. A precipitated hyperpolarized substrate prepared by the method of claim 1.

27. A redissolved hyperpolarized substrate prepared by the method of claim 6.

28. A biocompatible contrast agent comprising the precipitated hyperpolarized substrate of claim 26.

29. A method for diagnosing a disease or monitoring progress of treatment of a disease in a subject, the method comprising:
(a) administering the contrast agent of claim 28 to the subject; and
(b) performing imaging on the subject,
wherein performing imaging enables visualization of the precipitated hyperpolarized substrate or redissolved hyperpolarized substrate in the subject.

30.-33. (canceled)

34. The method of claim 29, wherein the disease comprises cancer, cardiovascular disease, or a metabolic disorder.

35. The method of claim 34, wherein the cancer comprises prostate cancer, breast cancer, or brain cancer.

36. The method of claim 34, wherein the metabolic disorder comprises diabetes, pyruvate dehydrogenase complex deficiency, or pyruvate carboxylase deficiency.

37. The method of claim 29 wherein the imaging is magnetic resonance imaging (MRI).

38.-39. (canceled)
